SEQUENCE LISTING (45 sequences)

SEQ ID NO: 1 - RNA, 32 nt, Artificial Sequence
  3′ Azido crgRNA, truncated. a synthetic oligonucleotide.
  misc_feature (1)..(20): n is a, c, g, or u
nnnnnnnnnnnnnnnnnnnnguuuuagagcua

SEQ ID NO: 2 - RNA, 36 nt, Artificial Sequence
  5′-alkynyl 3′-amino tracrg1RNA, a synthetic oligonucleotide.
uagcaaguuaaaauaaggcuaguccguuaucaacuu

SEQ ID NO: 3 - RNA, 68 nt, Artificial Sequence
  3′-Amino RNA segment comprising crgRNA and tracrg1RNA joined by a nNt-linker between a32 and u33.
  misc_feature (1)..(20): n is a, c, g, or u
nnnnnnnnnnnnnnnnnnnnguuuuagagcuauagcaaguuaaaauaaggcuaguccguuaucaacuu

SEQ ID NO: 4 - RNA, 68 nt, Artificial Sequence; identical to SEQ ID NO: 3
  3′-Azido RNA segment comprising crgRNA and tracrg1RNA joined by a nNt-linker between a32 and u33.
  misc_feature (1)..(20): n is a, c, g, or u

SEQ ID NO: 5 - RNA, 22 nt, Artificial Sequence
  5′-cyclooctynyl tracrg2RNA, a synthetic oligonucleotide.
aaguggcaccgagucggugcuu

SEQ ID NO: 6 - RNA, 90 nt, Artificial Sequence
  l2gRNA joined by two non-nucleotide linkers between a32 and u33, and u68 and a69, respectively.
  misc_feature (1)..(20): n is a, c, g, or u
nnnnnnnnnnnnnnnnnnnnguuuuagagcuauagcaaguuaaaauaaggcuaguccguuaucaacuuaaguggcaccgagucggugcuu

SEQ ID NO: 7 - RNA, 22 nt, Artificial Sequence
  5′-alkynyl tracrg2RNA, a synthetic oligonucleotide.
aaguggcaccgagucggugcuu

SEQ ID NO: 8 - RNA, 90 nt, Artificial Sequence; identical to SEQ ID NO: 6
  l2gRNA joined by two non-nucleotide linkers between a32 and u33, and u68 and a69, respectively.
  misc_feature (1)..(20): n is a, c, g, or u

SEQ ID NO: 9 - RNA, 22 nt, Artificial Sequence
  5′-triphenylphosphine l tracrg2RNA, a synthetic oligonucleotide.
aaguggcaccgagucggugcuu

SEQ ID NO: 10 - RNA, 90 nt, Artificial Sequence; identical to SEQ ID NO: 6
  l2gRNA joined by two non-nucleotide linkers between a32 and u33, and u68 and a69, respectively.
  misc_feature (1)..(20): n is a, c, g, or u

SEQ ID NO: 11 - RNA, 22 nt, Artificial Sequence
  5′-alkynyl, 3′-amino tracrg2RNA, a synthetic oligonucleotide.
aaguggcaccgagucggugcuu

SEQ ID NO: 12 - RNA, 90 nt, Artificial Sequence; identical to SEQ ID NO: 6
  3′-amino l2gRNA joined by two non-nucleotide linkers between a32 and u33, and u68 and a69, respectively.
  misc_feature (1)..(20): n is a, c, g, or u

SEQ ID NO: 13 - RNA, 90 nt, Artificial Sequence; identical to SEQ ID NO: 6
  3′-conjugates of l2gRNA joined by two non-nucleotide linkers between a32 and u33, and u68 and a69, respectively.
  misc_feature (1)..(20): n is a, c, g, or u

SEQ ID NO: 14 - RNA, 32 nt, Artificial Sequence; identical to SEQ ID NO: 1
  5′-Amino, 3′-alkynyl crgRNA, truncated, a synthetic oligonucleotide.
  misc_feature (1)..(20): n is a, c, g, or u

SEQ ID NO: 15 - RNA, 36 nt, Artificial Sequence; identical to SEQ ID NO: 2
  5′-Amino 3′-azido tracrg1RNA, a synthetic oligonucleotide.

SEQ ID NO: 16 - DNA, 23 bp, Artificial Sequence
  5′-alkynyl tracrg2RNA, a synthetic oligonucleotide.
aaguggcaccgagucggugcuut

SEQ ID NO: 17 - DNA, 59 bp, Artificial Sequence
  5′-amino l-tracrgRNA comprising a nNt-linker between u36 and a37, a synthetic oligonucleotide.
uagcaaguuaaaauaaggcuaguccguuaucaacuuaaguggcaccgagucggugcuuc

SEQ ID NO: 18 - DNA, 59 bp, Artificial Sequence; identical to SEQ ID NO: 17
  5′-azido l-tracrgRNA comprising a nNt-linker between u36 and a37, a synthetic oligonucleotide.

SEQ ID NO: 19 - DNA, 91 bp, Artificial Sequence
  l2gRNA joined by two non-nucleotide linkers between a32 and u33, and u68 and a69, respectively.
  misc_feature (1)..(20): n is a, c, g, t or u
nnnnnnnnnnnnnnnnnnnnguuuuagagcuauagcaaguuaaaauaaggcuaguccguuaucaacuuaaguggcaccgagucggugcuuc

SEQ ID NO: 20 - DNA, 120 bp, Artificial Sequence
  segRNA joined by two non-nucleotide linkers between a32 and u33, and u68 and a69, respectively.
  misc_feature (1)..(20): n is a, c, g, t or u
  misc_feature (91)..(120): n is a, c, g, t or u
nnnnnnnnnnnnnnnnnnnnguuuuagagcuauagcaaguuaaaauaaggcuaguccguuaucaacuuaaguggcaccgagucggugcuunnnnnnnnnnnnnnnnnnnnnnnnnnnnnn

SEQ ID NO: 21 - DNA, 30 bp, Artificial Sequence
  Complementary sequence of ssDNA adaptor in segRNA of Sequence No. 20..
  misc_feature (1)..(30): n is a, c, g, or t
nnnnnnnnnnnnnnnnnnnnnnnnnnnnnn

SEQ ID NO: 22 - DNA, 120 bp, Artificial Sequence; identical to SEQ ID NO: 20
  segRNA joined by two non-nucleotide linkers between a32 and u33, and u68 and a69, respectively. Only one strand of dsDNA adaptor segment is shown.
  misc_feature (1)..(20): n is a, c, g, t or u
  misc_feature (91)..(120): n is a, c, g, t or u

SEQ ID NO: 23 - DNA, 103 bp, Artificial Sequence
  segRNA joined by two non-nucleotide linkers between a32 and u33, and u68 and a69, respectively. The dsDNA is processed with an endouclease to give a sticky end. Only the covalently joined strand of dsDNA adaptro is shown.
  misc_feature (1)..(20): n is a, c, g, t or u
  misc_feature (91)..(103): n is a, c, g, t or u
nnnnnnnnnnnnnnnnnnnnguuuuagagcuauagcaaguuaaaauaaggcuaguccguuaucaacuuaaguggcaccgagucggugcuunnnnnnnnnnnnn

SEQ ID NO: 24 - DNA, 147 bp, Artificial Sequence
  segRNA joined by two non-nucleotide linkers between a32 and u33, and u68 and a69, respectively. The dsDNA is a gene editing template. Only the covalently joined strand of the dsDNA template is shown. Incomplete sequence.
  misc_feature (1)..(20): n is a, c, g, t or u
  misc_feature (91)..(147): n is a, c, g, t or u
nnnnnnnnnnnnnnnnnnnnguuuuagagcuauagcaaguuaaaauaaggcuaguccguuaucaacuuaaguggcaccgagucggugcuunnnnnnnnnnnnnnnnnnnnnnnnnnnnnnnnnnnnnnnnnnnnnnnnnnnnnnnnn

SEQ ID NO: 25 - DNA, 115 bp, Artificial Sequence
  segRNA joined by two non-nucleotide linkers between a32 and u33, and u68 and a69, respectively. The ssDNA hybridized with the adaptor is a gene editing template. Only the covalently joined strand of ssDNA adaptor segment is shown. Incomplete sequence.
  misc_feature (1)..(20): n is a, c, g, t or u
  misc_feature (91)..(115): n is a, c, g, t or u
nnnnnnnnnnnnnnnnnnnnguuuuagagcuauagcaaguuaaaauaaggcuaguccguuaucaacuuaaguggcaccgagucggugcuunnnnnnnnnnnnnnnnnnnnnnnnn

SEQ ID NO: 26 - RNA, 44 nt, Artificial Sequence
  A two-segment nucleic acid comprising ADAR guide RNA′s specificity domain and 3′-segment of its recruiting domain, joined by a nNt-linker.
  misc_feature (27)..(44): n is a, c, g, or u
aauguuguucucgucuccucgacaccnnnnnnnnnnnnnnnnnn

SEQ ID NO: 27 - RNA, 69 nt, Artificial Sequence
  A three-segment ADAR guide RNA joined by two nNt-linkers between u23 and a24, and c51 and n52, respectively.
  misc_feature (52)..(69): n is a, c, g, or u
ggugucgagaagaggagaacaauauaauguuguucucgucuccucgacaccnnnnnnnnnnnnnnnnnn

SEQ ID NO: 28 - RNA, 32 nt, Artificial Sequence
  3′ Azido crgRNA, truncated, a synthetic oligonucleotide targeting eGFP.
cucgugaccacccugaccuaguuuuagagcua

SEQ ID NO: 29 - DNA, 64 bp, Artificial Sequence
  5′-alkynyl tracrgRNA.
uagcaaguuaaaauaaggcuaguccguuaucaacuugaaaaaguggcaccgagucggugcuuut

SEQ ID NO: 30 - DNA, 96 bp, Artificial Sequence
  lgRNA targeting eGFP and joined by a non-nucleotide linker between a32 and u33.
cucgugaccacccugaccuaguuuuagagcuauagcaaguuaaaauaaggcuaguccguuaucaacuugaaaaaguggcaccgagucggugcuuut

SEQ ID NO: 31 - RNA, 32 nt, Artificial Sequence; identical to SEQ ID NO: 28
  5′-Amino, 3′ azido crgRNA, truncated, a synthetic oligonucleotide targeting eGFP.

SEQ ID NO: 32 - DNA, 96 bp, Artificial Sequence; identical to SEQ ID NO: 30
  5′-Amino lgRNA targeting eGFP and joined by a non-nucleotide linkers between a32 and u33.

SEQ ID NO: 33 - DNA, 96 bp, Artificial Sequence; identical to SEQ ID NO: 30
  5′-Azido lgRNA targeting eGFP and joined by a non-nucleotide linker between a32 and u33.

SEQ ID NO: 34 - DNA, 51 bp, Artificial Sequence
  5′-Alkynyl ssDNA template for eGFP --> BFP editing.
ctggcccaccctcgtgaccaccctgagccacggggtgcagtgcttcagccg

SEQ ID NO: 35 - DNA, 147 bp, Artificial Sequence
  segRNA (eGFP) joined by two nNT-linkers, between c1 and c52, and a83 and u84, respectively. There is no bond between g51 and c52.
ctggcccaccctcgtgaccaccctgagccacggggtgcagtgcttcagccgcucgugaccacccugaccuaguuuuagagcuauagcaaguuaaaauaaggcuaguccguuaucaacuugaaaaaguggcaccgagucggugcuuut

SEQ ID NO: 36 - DNA, 147 bp, Artificial Sequence
  segRNA (eGFP) joined by two nNT-linkers, between c1 and c52, and a83 and u84, respectively. There is no bond between g51 and c52.
cggctgaagcactgcaccccgtggctcagggtggtcacgagggtgggccagcucgugaccacccugaccuaguuuuagagcuauagcaaguuaaaauaaggcuaguccguuaucaacuugaaaaaguggcaccgagucggugcuuut

SEQ ID NO: 37 - RNA, 36 nt, Artificial Sequence; identical to SEQ ID NO: 2
  5′-alkynyl 3′-amino tracrg1RNA, a synthetic oligonucleotide.

SEQ ID NO: 38 - RNA, 68 nt, Artificial Sequence
  3′-Amino RNA segment comprising crRNA (eGFP) and tracrg1RNA joined by a nNt-linker between a32 and u33.
cucgugaccacccugaccuaguuuuagagcuauagcaaguuaaaauaaggcuaguccguuaucaacuu

SEQ ID NO: 39 - RNA, 68 nt, Artificial Sequence; identical to SEQ ID NO: 38
  3′-Azido RNA segment comprising crRNA (eGFP) and tracrg1RNA joined by a nNt-linker between a32 and u33.

SEQ ID NO: 40 - RNA, 68 nt, Artificial Sequence; identical to SEQ ID NO: 38
  3′-Azido RNA segment comprising crgRNA (eGFP) and tracrg1RNA joined by a nNt-linker between a32 and u33.

SEQ ID NO: 41 - DNA, 23 bp, Artificial Sequence
  5′-alkynyl tracrg2RNA, a synthetic oligonucleotide.
aaguggcaccgagucggugcuuc

SEQ ID NO: 42 - DNA, 91 bp, Artificial Sequence
  l2gRNA targeting eGFP, joined by two non-nucleotide linkers between a32 and u33, and u68 and a69, respectively.
cucgugaccacccugaccuaguuuuagagcuauagcaaguuaaaauaaggcuaguccguuaucaacuuaaguggcaccgagucggugcuuc

SEQ ID NO: 43 - DNA, 74 bp, Artificial Sequence
  5′-Alkynyl tracrg2RNA-ssDNA (eGFP).
aaguggcaccgagucggugcuuuctggcccaccctcgtgaccaccctgagccacggggtgcagtgcttcagccg

SEQ ID NO: 44 - DNA, 142 bp, Artificial Sequence
  segRNA targeting sGFP, joined by two non-nucleotide linkers between a32 and u33, and u68 and a69, respectively.
cucgugaccacccugaccuaguuuuagagcuauagcaaguuaaaauaaggcuaguccguuaucaacuuaaguggcaccgagucggugcuuuctggcccaccctcgtgaccaccctgagccacggggtgcagtgcttcagccg

SEQ ID NO: 45 - DNA, 142 bp, Artificial Sequence
  segRNA targeting sGFP, joined by two non-nucleotide linkers between a32 and u33, and u68 and a69, respectively.
cucgugaccacccugaccuaguuuuagagcuauagcaaguuaaaauaaggcuaguccguuaucaacuuaaguggcaccgagucggugcuuucggctgaagcactgcaccccgtggctcagggtggtcacgagggtgggccag

(19) United States
(12) Patent Application Publication
(10) Pub. No.: US 2023/0167441 A1
(43) Pub. Date: Jun. 1, 2023

(54) Segmented Nucleic Acids

(71) Applicant: Minghong Zhong, Sellersville, PA (US)

(72) Inventor: Minghong ZHONG, Sellersville, PA (US)

(21) Appl. No.: 17/839,539

(22) Filed: Jun. 14, 2022

Related U.S. Application Data
(60) Provisional application No. 63/284,025, filed on Nov. 30, 2021.

(51) Int. Cl. C12N 15/113; C12N 9/22

(52) U.S. Cl. CPC C12N 15/113; C12N 9/22

(57) ABSTRACT

Provided herein are processes and methods for preparation of segmented nucleic acids and segmented nucleic acid conjugates comprising at least two non-nucleotide linkers, and their RNP complexes with RNA guided gene editing proteins including CRISPR Cas proteins and ADAR enzymes. Also disclosed are the uses of the compositions comprising segmented nucleic acids or segmented nucleic acid conjugates as medicinal agents for treatment of diseases.

### CROSS REFERENCE TP RELATED APPLICATIONS

The present application claims the benefits of U.S. Provisional Application Ser. No. 63/284,025, filed Nov. 30, 2021, the entire said invention being incorporated herein by reference.

### TECHNICAL FIELD OF THE INVENTION

The present invention relates to segmented nucleic acids, their syntheses and uses as component(s) of therapeutics. The segmented nucleic acids each comprise at least two segments joined together by non-nucleotide linkers, and optionally are conjugated with other molecules for better drug properties such as cell-selective delivery. In particular, the disclosure relates to segmented nucleic acids and nucleic acid conjugates, their RNP complexes with RNA guided gene editing proteins such as CRISPR Cas9, nCas9, dCas9, fusion proteins, other Class 2 CRISPR endonucleases and ADAR, and their uses as medicinal agents for treatment of diseases.

### BACKGROUND OF THE INVENTION

Natural nucleic acids are polymers composed of nucleotides joined together by phosphate diester bonds. It is known that not all the phosphate diester bonds are required for the biological functions of nucleic acids. An extreme example is peptide nucleic acids, which are synthesized by amide coupling. Long oligonucleotides have diverse applications including uses as therapeutic nucleic acids, mRNA vaccines against COVID-19 as a prominent example, and gRNAs in gene editing, but syntheses, purifications and analytical characterizations of long RNA have been persistently challenging.

We disclosed non-nucleotide linkers for functional long nucleic acids. In particular, such linkers can replace the tetraloop in a gRNA between crRNA and tracrRNA and nucleotides void of interactions with Cas9 to give a chemically ligated functional gRNA (lgRNA). This not only makes manufacturing any long gRNA cost-effective, but also gives access to high quality validated full-length products with much fewer synthetic errors at the critical spacer segment than sgRNA, and enables cost-effective various chemical modifications for better efficacy and selectivity, stability, targeted delivery by molecular tagging, and so forth. Synthetic errors at the critical spacer segment cause extra guide-dependent off-target cleavage. Triazole has been delicately introduced into ribozymes, and the resulting products were reported to be biologically active. DNA incorporated with a triazole linker was disclosed as an effective template for DNA synthesis. Therefore, segmented nucleic acids are important family of nucleic acid analogues.

Syntheses of these segmented nucleic acids are convergent, and thus highly efficient; however, till present, efficient syntheses of nucleic acids with two and more than two non-nucleotide linkers are still lacking.

In addition, long nucleic acids form various secondary structures, and only some of these structures can bind the proteins such as RNA guided endonucleases to form fully functional RNA-protein (RNP) complexes. This also leads to great challenges in their separations/purifications and analytical characterizations.

This invention pertains to chemically ligated nucleic acids including guide RNA oligonucleotides (lgRNA), and discloses a highly efficient chemical method for preparation of segmented nucleic acids with two or more than two non-nucleotide linkers.

In addition, this invention pertains to applications of non-nucleotide linkers to enhancing or regulating the function of the resulting nucleic acids by altering the population of their secondary structures and/or introducing additional molecular interactions including hydrogen bonds.

This invention further pertains to the uses of segmented nucleic acids and segmented nucleic acid conjugates as component(s) of compositions for gene editing, and in particular, for treatment of diseases.

### SUMMARY OF THE INVENTION

The present invention pertains to segmented nucleic acids, their syntheses, and their uses as component(s) of therapeutics. The segmented nucleic acids each comprise two or more than two segments joined together by non-nucleotide linkers, and optionally are conjugated with other molecules for better drug properties such as cell-selective delivery.

In some aspects, the invention provides segmented nucleic acids comprising non-nucleotide linkers formed by chemical ligations, and the non-nucleotide linkers have little-to-no effects of decreasing the function of the resulting nucleic acids.

In some aspects, the invention provides segmented nucleic acids comprising non-nucleotide linkers formed by chemical ligations, and non-nucleotide linkers enhance the function of the resulting nucleic acids by altering the population of their secondary structures and/or introducing additional molecular interactions including hydrogen bonds.

In some aspects, this invention pertains to applications of non-nucleotide linkers to enhancing the function of lgRNAs by altering the population of their secondary structures and/or introducing additional molecular interactions including hydrogen bonds.

In some aspects, the invention provides segmented nucleic acids comprising non-nucleotide linkers formed by chemical ligations, and the non-nucleotide linkers have one or more chemical moieties for temporal control and/or cell-selective regulations of the function of the resulting nucleic acids. The chemical moieties include photocleavable functions, a disulfide bond, and functions cleavable in specific cells and in certain cellular microenvironments.

In some aspects, this invention pertains to chemically ligated nucleic acids including guide RNA oligonucleotides (lgRNA), and discloses a highly efficient chemical method for preparation of segmented nucleic acids with two non-nucleotide linkers.

In some aspects, the invention provides methods for producing nucleic acid molecules, comprising: (a) separately synthesizing three or more nucleic acid segments equipped with chemical functions for one-pot orthogonal or sequential chemical ligations, (b) contacting the nucleic acid segments with each other under conditions that allow for chemical ligations of the 3′ terminus of one nucleic acid segment to the 5′ terminus of a second nucleic acid segment to produce a segmented nucleic acid molecule.

The invention also includes methods for producing chemically ligated single molecule guide RNAs for CRISPR mediated gene editing. These methods comprise: (a) separately synthesizing three or more nucleic acid segments equipped with chemical functions for one-pot orthogonal or sequential chemical ligations, (b) contacting the nucleic acid segments with each other under conditions that allow for chemical ligations of the 3′ terminus of one nucleic acid segment to the 5′ terminus of a second nucleic acid segment to produce a segmented guide RNA.

In some aspects, the invention provides segmented nucleic acids further comprising one or more molecules for cell targeting, each conjugated via a non-nucleotide linker.

In some aspects, the invention is directed to methods comprising: (a) separately synthesizing nucleic acid segments and cell-targeting ligands each equipped with chemical functions for one-pot orthogonal or sequential chemical ligations, (b) contacting the nucleic acid segments with each other under conditions that allow for chemical ligations of the 3′ terminus of one nucleic acid segment to the 5′ terminus of a second nucleic acid segment to produce a segmented nucleic acid molecule and (c) contacting the formed segmented nucleic acid molecule with cell-targeting ligands to produce a segmented nucleic acid-ligand conjugate.

In some aspects, the invention is directed to methods comprising: (a) separately synthesizing nucleic acid segments and peptides each equipped with chemical functions for one-pot orthogonal or sequential chemical ligations, (b) contacting the nucleic acid segments with each other under conditions that allow for chemical ligations of the 3′ terminus of one nucleic acid segment to the 5′ terminus of a second nucleic acid segment to produce a segmented nucleic acid molecule and (c) contacting the formed segmented nucleic acid molecule with peptides to produce a segmented nucleic acid-peptides conjugate.

In some aspects, the invention is directed to methods comprising: (a) separately synthesizing nucleic acid segments and proteins each equipped with chemical functions for one-pot orthogonal or sequential chemical ligations, (b) contacting the nucleic acid segments with each other under conditions that allow for chemical ligations of the 3′ terminus of one nucleic acid segment to the 5′ terminus of a second nucleic acid segment to produce a segmented nucleic acid molecule and (c) contacting the formed segmented nucleic acid molecule with proteins to produce a segmented nucleic acid-protein conjugate.

In some aspects, the invention is directed to methods comprising: (a) separately synthesizing nucleic acid segments and polyethylene glycols (PEG) each equipped with chemical functions for one-pot orthogonal or sequential chemical ligations, (b) contacting the nucleic acid segments with each other under conditions that allow for chemical ligations of the 3′ terminus of one nucleic acid segment to the 5′ terminus of a second nucleic acid segment to produce a segmented nucleic acid molecule and (c) contacting the formed segmented nucleic acid molecule with PEGs to produce a segmented nucleic acid-PEG conjugate.

In some aspects, the invention is directed to methods comprising: (a) separately synthesizing nucleic acid segments and polymers each equipped with chemical functions for one-pot orthogonal or sequential chemical ligations, (b) contacting the nucleic acid segments with each other under conditions that allow for chemical ligations of the 3′ terminus of one nucleic acid segment to the 5′ terminus of a second nucleic acid segment to produce a segmented nucleic acid molecule and (c) contacting the formed segmented nucleic acid molecule with polymers to produce a segmented nucleic acid-polymer conjugate.

The invention also includes methods for producing chemically ligated single molecule guide RNA-ssDNA conjugates for CRISPR mediated precise gene editing. These methods comprise: (a) separately synthesizing three or more nucleic acid segments equipped with chemical functions for one-pot orthogonal or sequential chemical ligations, (b) contacting the nucleic acid segments with each other under conditions that allow for chemical ligations of the 3′ terminus of one nucleic acid segment to the 5′ terminus of a second nucleic acid segment to produce a segmented nucleic acid molecule. The 5′ terminal segment of the resulting ligated conjugate is an ssDNA of DNA repair template comprising the gene editing sequence flanked with two homology arms.

In one embodiment, the 5′ terminus of ssDNA is ligated to the 5′ terminus of ligated guide RNA.

In another embodiment, the 3′ terminus of ssDNA is ligated to the 5′ terminus of ligated guide RNA.

The invention further includes methods for producing chemically ligated single molecule guide RNA-ssDNA conjugates for CRISPR mediated precise gene editing. These methods comprise: (a) separately synthesizing three or more nucleic acid segments equipped with chemical functions for one-pot orthogonal or sequential chemical ligations, (b) contacting the nucleic acid segments with each other under conditions that allow for chemical ligations of the 3′ terminus of one nucleic acid segment to the 5′ terminus of a second nucleic acid segment to produce a segmented nucleic acid molecule. The 3′ terminal segment of the resulting ligated conjugate is an ssDNA DNA repair template comprising the gene editing sequence flanked with two homology arms.

In one embodiment, the 5′ terminus of ssDNA is ligated to the 3′ terminus of ligated guide RNA.

The invention still further includes methods for producing chemically ligated single molecule guide RNAs armed with an ssDNA template for CRISPR mediated precise gene editing. These methods comprise: (a) separately synthesizing three or more nucleic acid segments equipped with chemical functions for one-pot orthogonal or sequential chemical ligations, (b) contacting the nucleic acid segments with each other under conditions that allow for chemical ligations of the 3′ terminus of one nucleic acid segment to the 5′ terminus of a second nucleic acid segment to produce a segmented nucleic acid molecule. The 3′ terminal segment comprises one RNA segment and one DNA segment joined by a phosphate diester bond or a phosphoramidate bond between the 3′ terminus of the RNA segment and the 5′ terminus of the DNA segment, and the DNA segment is a DNA repair template comprising the gene editing sequence flanked with two homology arms.

The invention also includes methods for producing chemically ligated single molecule guide RNAs armed with an adaptor ssDNA for CRISPR mediated gene editing. These methods comprise: (a) separately synthesizing three or more nucleic acid segments equipped with chemical functions for one-pot orthogonal or sequential chemical ligations, (b) contacting the nucleic acid segments with each other under conditions that allow for chemical ligations of the 3′ terminus of one nucleic acid segment to the 5′ terminus of a second nucleic acid segment to produce a segmented nucleic acid molecule. Either 5′ terminal segment or 3′ terminal segment is an adaptor ssDNA complementary to a cargo DNA molecule for gene therapy.

Some of these segmented nucleic acids or their conjugates form RNP complexes with proteins such as CRISPR Cas9, nCas9, dCas9 and fusion proteins, other Class 2 CRISPR endonucleases and ADAR, and the resulting RNP complexes are used as medicinal agents for treatment of diseases.

The invention further includes cells containing one or more segmented nucleic acids or their conjugates and cells made by methods set out herein. For example, the invention includes cells into which one or more segmented nucleic acids or their conjugates have been introduced with or without proteins such as Cas9, nCas9, nCas9 fusion proteins, dCas9, dCas9 fusion proteins, other Class 2 CRISPR endonucleases and ADAR thereof. The invention further includes cells containing segmented nucleic acids or their conjugates and mRNA encoding the proteins such as Cas9, nCas9, nCas9 fusion proteins, dCas9 and fusion proteins, other Class 2 CRISPR endonucleases and ADAR thereof, as well as cells that have been modified by methods of the invention (e.g., cells that have undergone DNA cleavage(s) and modification(s) at the target site(s)) that either contain or no longer contain one or more segmented nucleic acids.

### BRIEF DESCRIPTION OF THE DRAWINGS

FIG. 1: shows LC/UV chromatogram of eGFP-targeting lgRNA by ESI-LCMS.

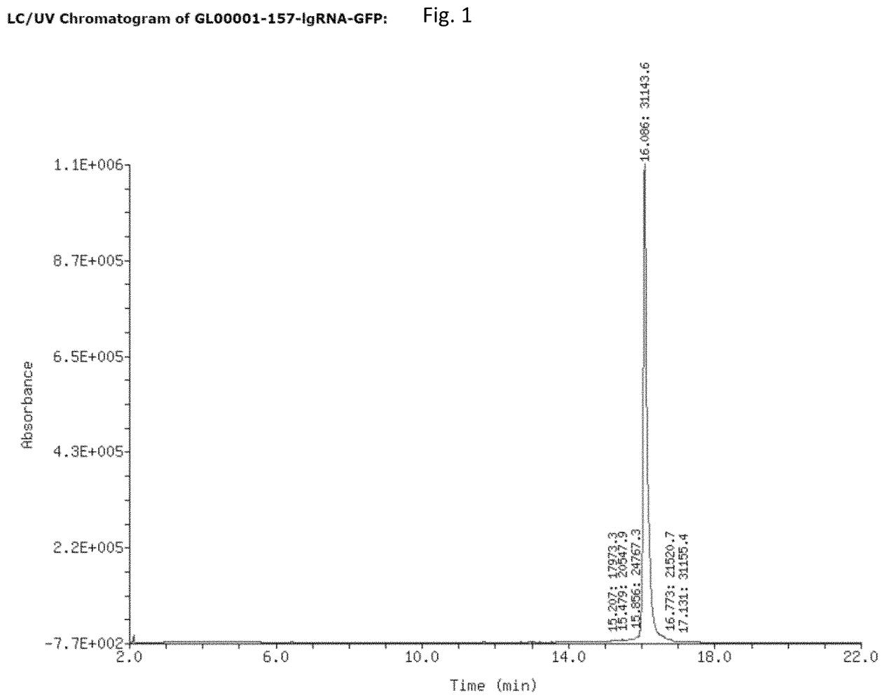

FIG. 2: shows molecule mass and intensity of each peak in the chromatogram.

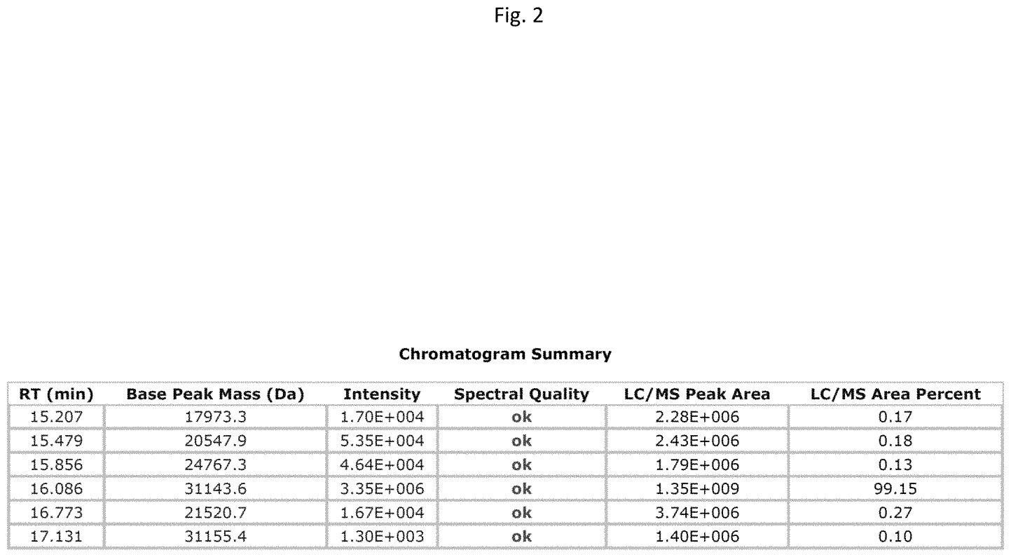

FIG. 3: shows schematic structures of lgRNA with one non-nucleotide linker and l2gRNA with two non-nucleotide linkers (top), and a gel image from in vitro cleavage assays of lgRNA and l2gRNA (bottom). The non-nucleotide linkers allow for a cis-configuration or the same orientation of their two side chains, respectively.

FIG. 4: shows LC/UV chromatogram of eGFP-targeting 5′-amino lgRNA (direct injection/without HPLC separation) by ESI-LCMS.

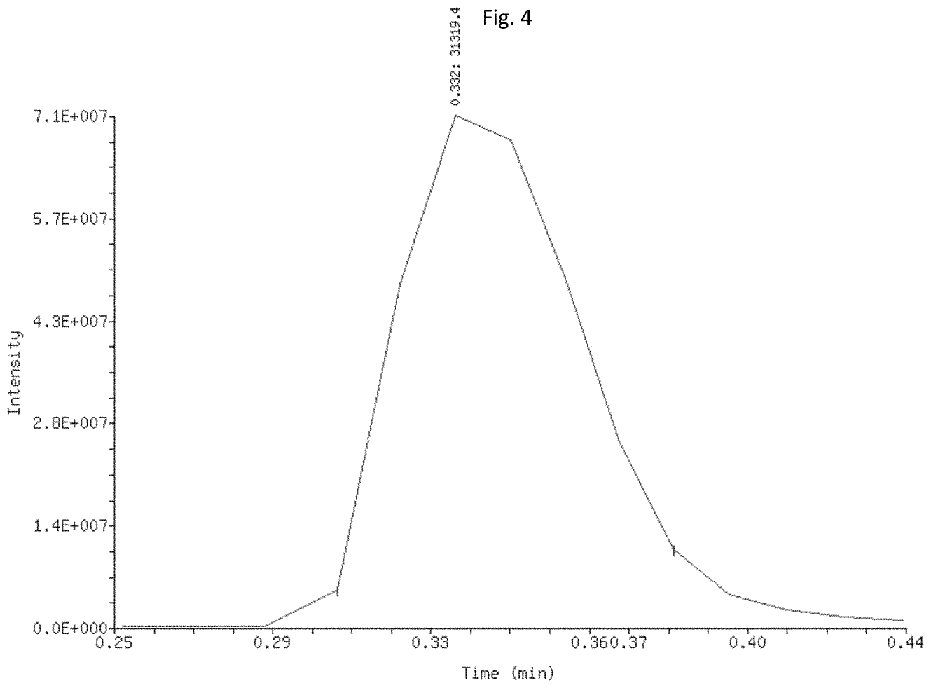

FIG. 5: shows charge states of molecular ion and deconvoluted mass of the sample in FIG. 4.

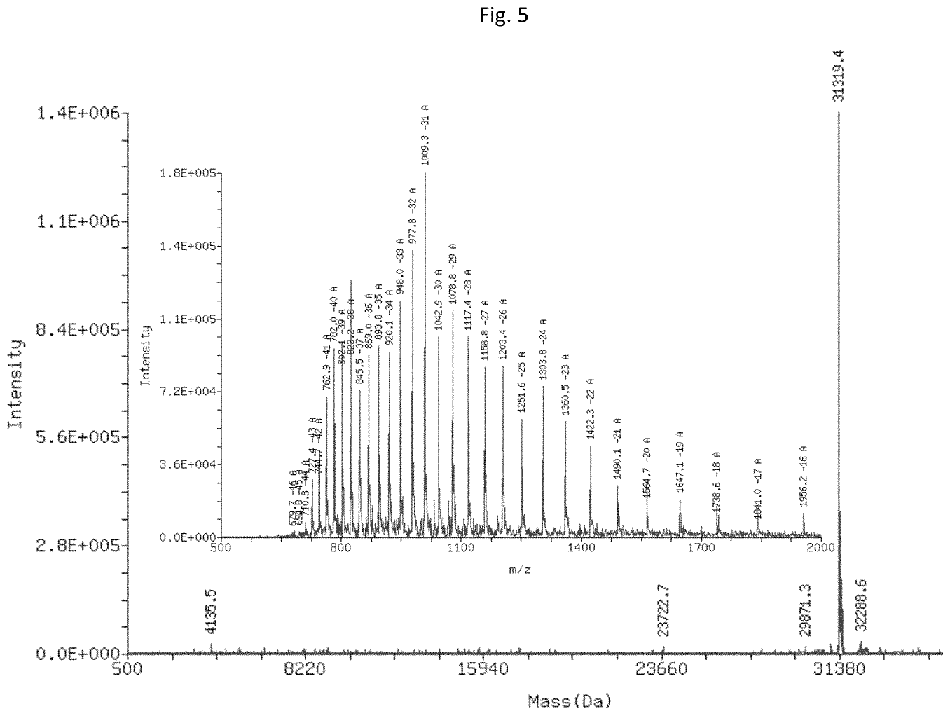

FIG. 6: shows a gel image from in vitro cleavage assays of segRNAs in comparison with lgRNA and l2gRNA.

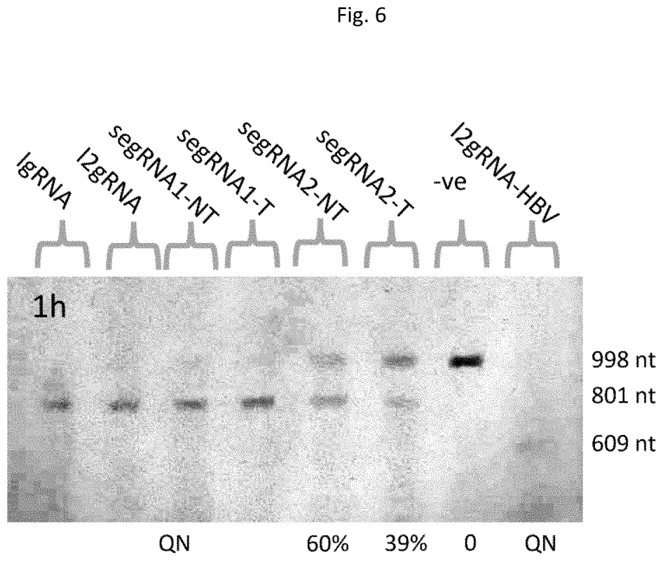

### DETAILED DESCRIPTION OF THE INVENTION

An aspect of the invention is directed to methods for production of chemically ligated segmented nucleic acids.

One embodiment of the invention is the use of a nucleic acid segment containing an amino function and an alkynyl function or containing an amino function and a phosphino function for sequential ligations by activation of the amino to an azido by a diazotransfer reaction with fluorosulfuryl azide after a chemical ligation step of the alkynyl or phosphino with an azide. The newly formed azido reacts with a second nucleic acid segment containing an amino function and an alkynyl function or an amino function and a phosphino function. These steps can be repeated for synthesis of a multiple-segmented nucleic acid.

One embodiment of the invention is the use of a nucleic acid segment containing an amino function and an alkynyl function for sequential ligations by activation of the amino to an azido by a diazotransfer reaction with fluorosulfuryl azide after a chemical ligation step of the alkynyl with an azide. The newly formed azido reacts with a second nucleic acid segment containing an amino function and an alkynyl function. These steps can be repeated for synthesis of a multiple-segmented nucleic acid.

One embodiment of the invention is the use of a nucleic acid segment containing an alkynyl function and at least one amino function for sequential ligations by activation of said amino groups to azido groups by a diazotransfer reaction with fluorosulfuryl azide after a chemical ligation step of the alkynyl with an azide.

Another embodiment of the invention is the use of a nucleic acid segment containing an amino function and an azido for sequential ligations by activation of the amino to an azido by a diazotransfer reaction with fluorosulfuryl azide after a chemical ligation step of the azido function with an alkyne. The newly formed azido reacts with a second nucleic acid segment containing an alkynyl function to provide a three-segment nucleic acid.

One embodiment of the invention is synthesis of a multiple-segmented RNA by the above sequential ligations.

Another embodiment of the invention is synthesis of a multiple-segmented DNA by the above sequential ligations.

Another embodiment of the invention is synthesis of a multiple-segmented nucleic acid comprising both DNA and RNA by the above sequential ligations.

Yet another embodiment of the invention is synthesis of multiple-segmented nucleic acid conjugates comprising DNA and/or RNA and other chemical moieties such as fluorescent dyes, polypeptides, carbohydrates, lipids, PEG and synthetic polymers, by the above sequential ligations.

One embodiment of the invention is synthesis of a multiple segmented ribozyme.

One embodiment of the invention is synthesis of a multiple segmented aptamer and riboswitch.

One embodiment of the invention is synthesis of a multiple segmented guide RNA of CRISPR-Cas.

One embodiment of the invention is synthesis of a multiple segmented guide RNA to recruit endogenous RNA-specific adenosine deaminase (ADAR) for RNA editing.

Another embodiment of the invention is synthesis of a multiple-segmented circular RNA.

In one embodiment of the invention, the ligation reaction is CuAAC (A-2 and B-1), or SPAAC (A-2 and B-2; A2-and B-3, etc.) or Staudinger ligation (A-2 and B-4) between two nucleic acids.

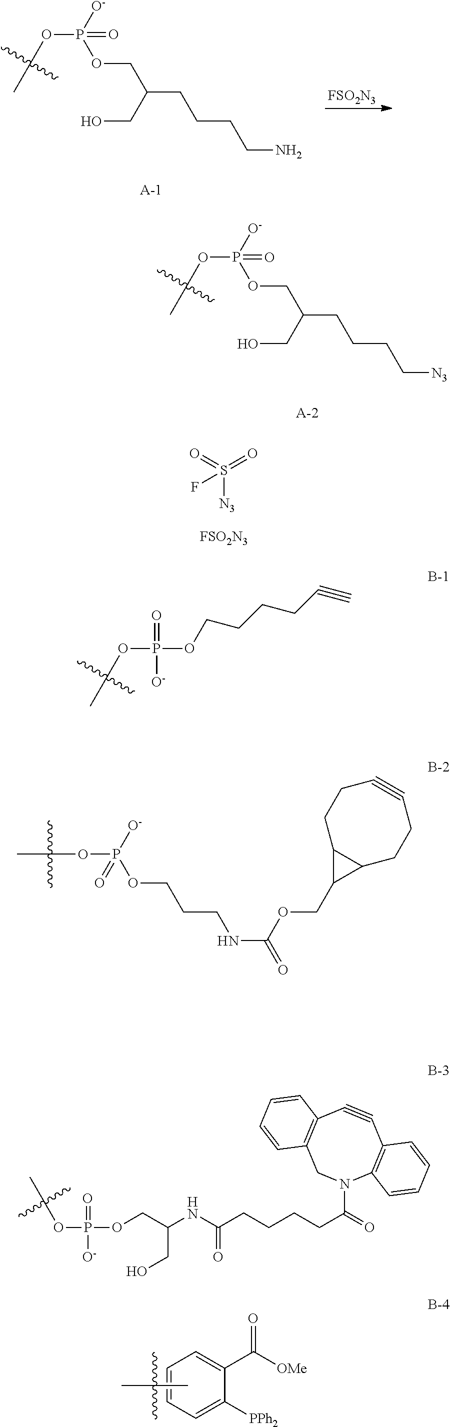

In another embodiment of the invention, sequential ligations comprise one type or other types of ligation reactions known to person having ordinary skill in the art. In another embodiment of the invention, said sequential ligations can be applied for synthesis of multiple-segmented nucleic acid conjugates.

In another embodiment of the invention, the ligation reactions include thiol-maleimide, strain promoted alkyne-azide cycloaddition (SPAAC)/Cu^{I}-catalyzed alkyne-azide cycloaddition (CuAAC) and inverse-electron-demand Diels-Alder (IEDDA) with a tetrazine. (See, e.g., U.S. Pat. No. 10,059,940 and US Patent Publication US 2016/0102322, the entire disclosures of which are incorporated herein by reference.)

One embodiment of the invention is preparation of a multiple-segment nucleic acid by sequential CuAACs, enabled by using an amine as a precursor for an azide, comprising following steps:
a) Synthesis of segment 1 of 8-200 nt in length containing azido modification at its 3′-end or a position close to its 3′-end;
b) Synthesis of segment 2 of 8-200 nt in length containing an alkynyl at its 5′-end or at a position close to its 5′-end, and an amino at its 3′-end or a position close to its 3′-end;
c) Ligation of said segment 1 and 2 by reaction between said azido and alkynyl to form a two-segmented nucleic acid linked by the resulting triazole;
d) Transformation of said amino of said two-segmented nucleic acid in step c) into an azido;
e) Ligation of azido two-segment nucleic acid in d) to another segment, containing both an amino and an alkynyl, between said azido and the alkynyl in said another segment;
f) Step d) and e) are repeated as needed to prepare a multiple-segmented nucleic acid;
g) Separate the segmented nucleic acid from unreacted shorter segments and chemical reagents,
   wherein, the products in steps a) to e) are optionally purified, or these purifications are skipped, and the crude final product is purified at step g).

One embodiment of the invention is preparation of a three-segment lgRNA by sequential CuAACs, SPAACs and/or Staudinger ligations, enabled by using an amine as a precursor for an azide, comprising three steps: Step 1. Click reaction of a 5′-amino-3′-azido nucleic acid with a 5′-alkynyl or 5′-phosphino nucleic acid; Step 2. Azide formation from the amine by a diazotransfer reaction with fluorosulfuryl azide; Step 3. Click reaction of the newly formed 5′-azido nucleic acid in step 2 with a 3′-alkynyl or 3′-phosphino nucleic acid.

One embodiment of the invention is preparation of a three-segment lgRNA by sequential CuAACs, SPAACs and/or Staudinger ligations, enabled by using an amine as a precursor for an azide, comprising three steps as follows: Step 1. Click reaction of a 3′-amino-5′-azido nucleic acid with a 3′-alkynyl or 3′-phosphino nucleic acid; Step 2. Azide formation from the amine in Step 1 by a diazotransfer reaction with fluorosulfuryl azide; Step 3. Click reaction of the formed 3′-azido nucleic acid in Step 2 with a 5′-alkynyl or 5′-phosphino nucleic acid.

Another embodiment of the invention is preparation of a three-segment lgRNA conjugate by sequential CuAACs, SPAACs and/or Staudinger ligations, enabled by using an amine as a precursor for an azide, comprising three steps as illustrated by sequential CuAACs and/or SPAACs: Step 1. Click reaction of a 5′-amino-3′-azido nucleic acid with a 5′-alkynyl nucleic acid; Step 2. Azide formation from the amine by a diazotransfer reaction with fluorosulfuryl azide; Step 3. Click reaction of the formed 5′-azido nucleic acid in step 2 with a 3′-alkynyl nucleic acid. Said 3′-alkynyl nucleic acid in step 3 contains at least one amino, which reacts with NHS esters or carboxylic acids to provide a three-segment lgRNA conjugate.

Another embodiment of the invention is preparation of a three-segment lgRNA conjugate by sequential CuAACs, SPAACs and/or Staudinger ligations, enabled by using an amine as a precursor for an azide, comprising three steps as illustrated by sequential CuAACs and/or SPAACs: Step 1. Click reaction of a 3′-amino-5′-azido nucleic acid with a 3′-alkynyl nucleic acid; Step 2. Azide formation from the amine by a diazotransfer reaction with fluorosulfuryl azide; Step 3. Click reaction of the formed 3′-azido nucleic acid in step 2 with a 5′-alkynyl nucleic acid. Said 5′-alkynyl nucleic acid in step 3 contains at least one amino, which reacts with NHS esters or carboxylic acids to provide a three-segment lgRNA conjugate.

The following are unlimited examples of formed corresponding non-nucleotide linkers.

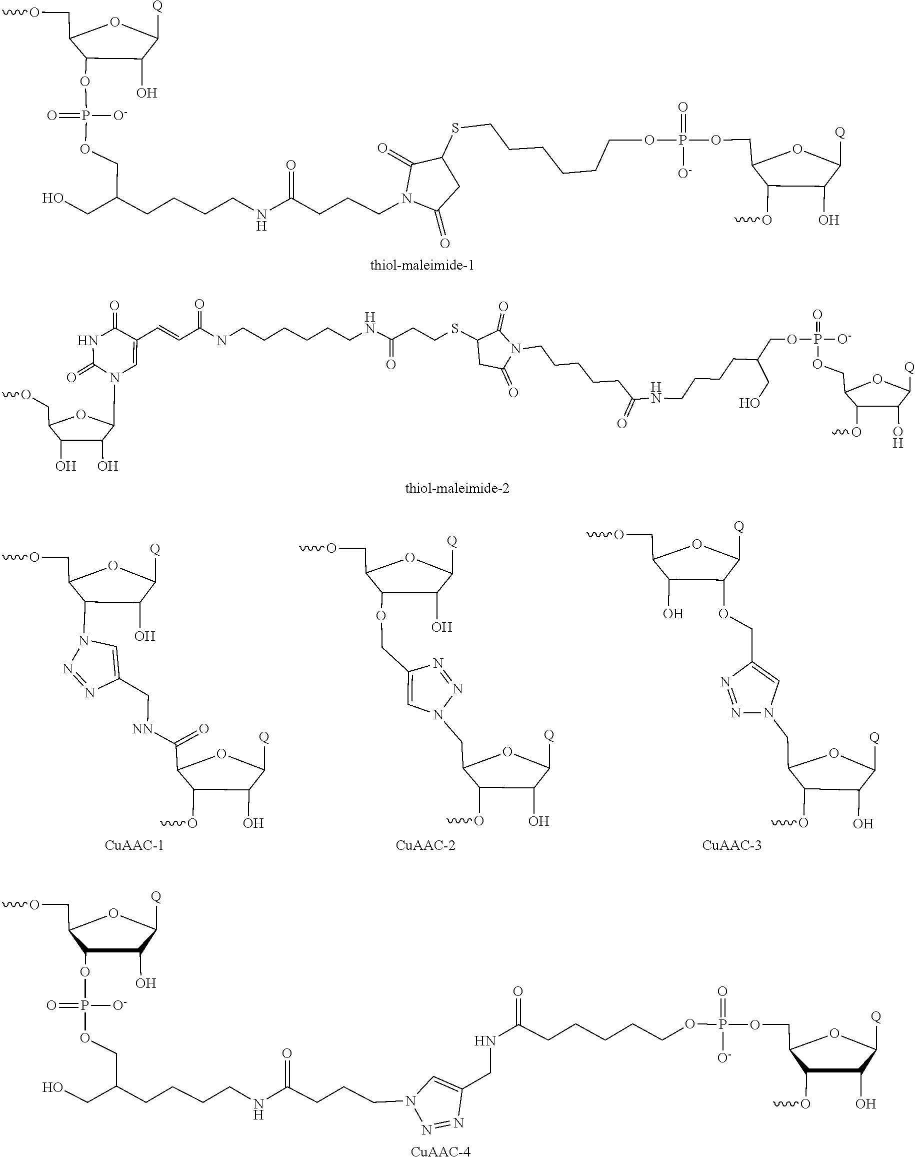

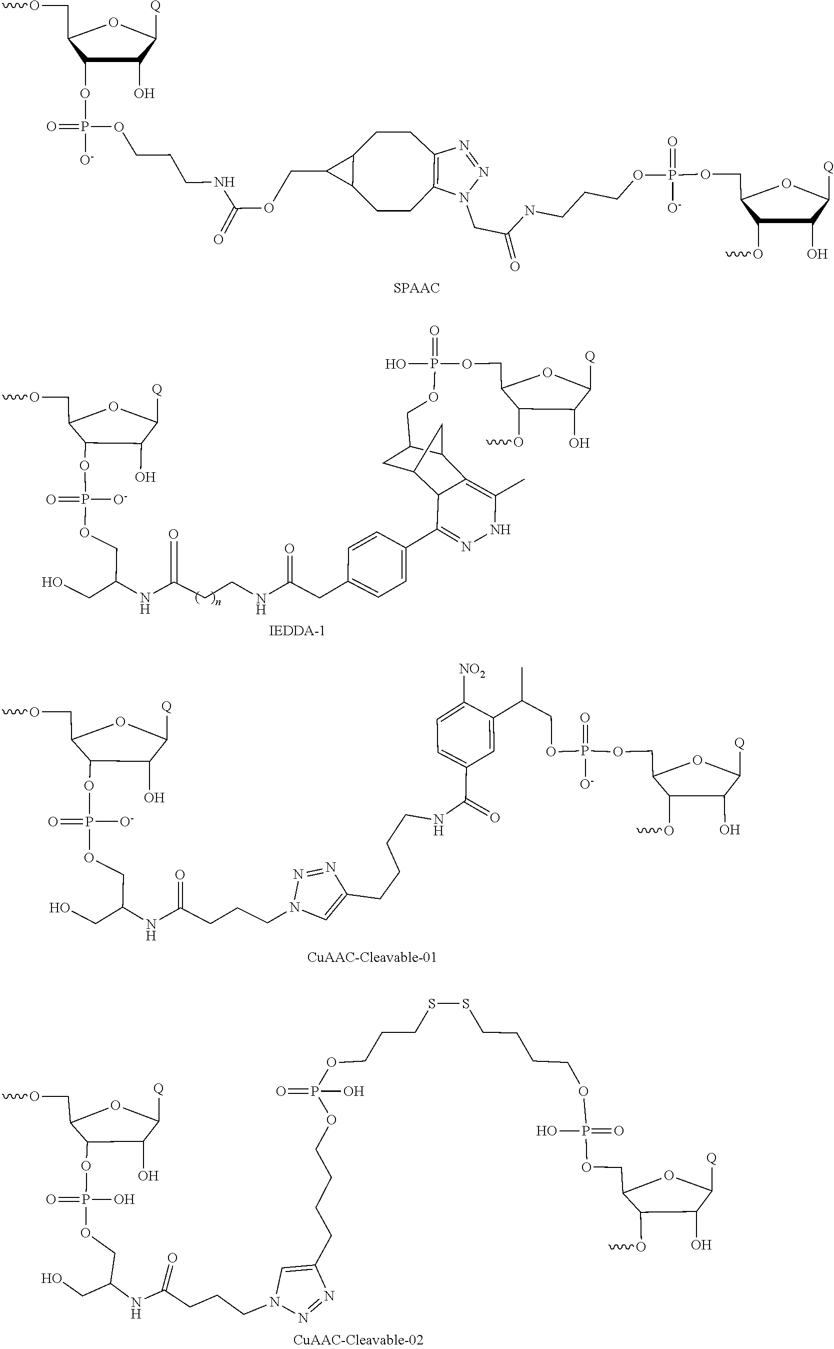

Definition

The definitions of terms used herein are consistent to those known to those of ordinary skill in the art, and in case of any differences the definitions are used as specified herein instead.

The term “nucleoside” as used herein refers to a molecule composed of a heterocyclic nitrogenous base, containing an N-glycosidic linkage with a sugar, particularly a pentose. An extended term of “nucleoside” as used herein also refers to acyclic nucleosides and carbocyclic nucleosides.

The term “nucleotide” as used herein refers to a molecule composed of a nucleoside monophosphate, di-, or triphosphate containing a phosphate ester at 5′-, 3′-position or both. The phosphate can also be a phosphonate or a phosphoramidate. The oxo in a nucleotide can be replaced by S or CF₂.

The term of “oligonucleotide” (ON) is herein used interchangeably with “polynucleotide”, “nucleotide sequence”, and “nucleic acid”, and refers to a polymeric form of nucleotides of any length, either deoxyribonucleotides or ribonucleotides, or analogs thereof. An oligonucleotide may comprise one or more modified nucleotides, which may be imparted before or after assembly of such a oligonucleotide. The sequence of nucleotides may be interrupted by non-nucleotide components.

The term of “modification” of nucleic acids includes but is not limited to (a) end modifications, e.g., 5′ end modifications or 3′ end modifications, (b) nucleobase (or “base”) modifications, including replacement or removal of bases, (c) sugar modifications, including modifications at the 2′, 3′, and/or 4′ positions, and (d) backbone modifications, including modification or replacement of the phosphodiester linkages. The term “modified nucleotide” generally refers to a nucleotide having a modification to the chemical structure of one or more of the base, the sugar, and the phosphodiester linkage or backbone portions, including nucleotide phosphates. (See, e.g., Ryan et al. US20160289675, the entire disclosure of which is incorporated herein by reference.)

The terms “Z” and “P” refer to the nucleotides, nucleobases, or nucleobase analogs developed by Steven Benner and colleagues as described for example in “Artificially expanded genetic information system: a new base pair with an alternative hydrogen bonding pattern” Yang, Z., Huffer, D., Sheng, P., Sismour, A. M. and Benner, S. A. Nucleic Acids Res. 2006, 34, 6095-101, the contents of which is hereby incorporated by reference in its entirety.

The terms “xA”, “xG”, “xC”, “xT”, or “x(A, G, C, T)” and “yA”, “yG”, “yC”, “yT”, or “y(A, G, C, T)” refer to nucleotides, nucleobases, or nucleobase analogs as described by Krueger et al. in “Synthesis and Properties of Size-Expanded DNAs: Toward Designed, Functional Genetic Systems”; Krueger et al. Acc. Chem. Res. 2007, 40, 141-50, the contents of which is hereby incorporated by reference in its entirety.

The term “Unstructured Nucleic Acid” or “UNA” refers to nucleotides, nucleobases, or nucleobase analogs as described in U.S. Pat. No. 7,371,580, the contents of which is hereby incorporated by reference in its entirety. An unstructured nucleic acid, or UNA, modification is also referred to as a “pseudo-complementary” nucleotide, nucleobase or nucleobase analog (See, e.g., Lahoud et al. Nucl. Acids Res. 1991, 36:10, 3409-19).

The terms “PACE” and “thioPACE” refer to internucleotide phosphodiester linkage analogs containing phosphonoacetate or thiophosphonoacetate groups, respectively. These modifications belong to a broad class of compounds comprising phosphonocarboxylate moiety, phosphonocarboxylate ester moiety, thiophosphonocarboxylate moiety and thiophosphonocarboxylate ester moiety. These linkages can be described respectively by the general formulae P(CR1R2)ₙCOOR and (S)—P(CR1R2)ₙCOOR wherein n is an integer from 0 to 6 and each of R1 and R2 is independently selected from the group consisting of H, an alkyl and substituted alkyl.

The term of “G-clamp” refers to a cytosine analogue capable of clamp-like binding to a guanine in helical nucleic acids by formation of additional hydrogen bonds (See, e.g., Lin et al. J. Am. Chem. Soc. 1998, 120, 33, 8531-8532; Wilds et al. Angew. Chem. Int. Ed. 2002, 41, 115-117).

The term of “CRISPR/Cas9” refers to the type II CRISPR-Cas system from Streptococcus pyogenes, Cas9 orthologues and variants. The type II CRISPR-Cas system comprises protein Cas9 and two noncoding RNAs (crRNA and tracrRNA). These two noncoding RNAs were further fused into one single guide RNA (sgRNA). The Cas9/sgRNA complex binds double-stranded DNA sequences that contain a sequence match to the first 17-20 nucleotides of the sgRNA and immediately before a protospacer adjacent motif (PAM). Once bound, two independent nuclease domains (HNH and RuvC) in Cas9 each cleaves one of the DNA strands 3 bases upstream of the PAM, leaving a blunt end DNA double stranded break (DSB).

The term of “off-target effects” refers to non-targeted cleavage of the genomic DNA target sequence by Cas9 in spite of imperfect matches between the gRNA sequence and the genomic DNA target sequence. Single mismatches of the gRNA can be permissive for off-target cleavage by Cas9. Off-target effects were reported for all the following cases: (a) same length but with 1-5 base mismatches; (b) off-target site in target genomic DNA has one or more bases missing (‘deletions’); (c) off-target site in target genomic DNA has one or more extra bases (‘insertions’).

The term of “guide RNA” (gRNA) refers to a synthetic fusion of crRNA and tracrRNA via a tetraloop (GAAA) (defined as sgRNA) or other chemical linkers such as an nNt-Linker (defined as lgRNA), and is used interchangeably with “chimeric RNA”, “chimeric guide RNA”, “single guide RNA” and “synthetic guide RNA”. The gRNA contains secondary structures of the repeat:anti-repeat duplex, stem loops 1-3, and the linker between stem loops 1 and 2 (See, e.g., Nishimasu et al. Cell 2014, 156, 935-949).

The term of “dual RNA” refers to hybridized complex of the short CRISPR RNAs (crRNA) and the trans-activating crRNA (tracrRNA). The crRNA hybridizes with the tracrRNA to form a crRNA:tracrRNA duplex, which is loaded onto Cas9 to direct the cleavage of cognate DNA sequences bearing appropriate protospacer-adjacent motifs (PAM).

The term of “lgRNA” refers to guide RNA (gRNA) joined by chemical ligations to form non-nucleotide linkers (nNt-linkers) between crgRNA and tracrgRNA, or at other sites.

The terms of “dual lgRNA”, “triple lgRNA” and “multiple lgRNA” refer to hybridized complexes of the synthetic guide RNA fused by chemical ligations via non-nucleotide linkers. Dual tracrgRNA is formed by chemical ligation between tracrgRNA1 and tracrgRNA2 (RNA segments of ˜30 nt), and crgRNA (˜30 nt) is fused with a dual tracrgRNA (1-tracrgRNA) to form a triple lgRNA duplex (l2gRNA), which is loaded onto Cas9 to direct the cleavage of cognate DNA sequences bearing appropriate protospacer-adjacent motifs (PAM). Each RNA segment can be readily accessible by chemical manufacturing and compatible to extensive chemical modifications.

The term “guide sequence” refers to the about 20 bp sequence within the guide RNA that specifies the target site and is herein used interchangeably with the terms “guide” or “spacer”. The term “tracr mate sequence” may also be used interchangeably with the term “direct repeat(s)”.

The term of “crgRNA” refers to crRNA equipped with chemical functions for conjugation/ligation and is used interchangeably with crRNA in an lgRNA comprising at least one non-Nucleotide linker. The oligonucleotide may be chemically modified close to its 3′-end, any one or several nucleotides, or for its full sequence.

The term of “tracrgRNA” refers to tracrRNA equipped with chemical functions for conjugation/ligation and is used interchangeably with tracrRNA in an lgRNA comprising at least one non-Nucleotide linker. The oligonucleotide may be chemically modified at any one or several nucleotides, or for its full sequence.

The term of “the protospacer adjacent motif (PAM)” refers to a DNA sequence immediately following the DNA sequence targeted by Cas9 in the CRISPR bacterial adaptive immune system, including NGG, NNNNGATT, NNAGAA, NAAAC, and others from different bacterial species where N is any nucleotide.

The term of “chemical ligation” refers to joining together synthetic oligonucleotides via an nNt-linker by chemical methods such as click ligation (the azide-alkyne reaction to produce a triazole linkage), thiol-maleimide reaction, and formations of other chemical groups.

The term of “complementary” refers to the ability of a nucleic acid to form hydrogen bond(s) with another nucleic acid sequence by either traditional Watson-Crick or other non-traditional types. Cas9 contains two nuclease domains, HNH and RuvC, which cleave the DNA strands that are complementary and noncomplementary to the 20 nucleotide (nt) guide sequence in crRNAs, respectively.

The term of “Hybridization” refers to a reaction in which one or more polynucleotides form a complex that is stabilized via hydrogen bonding between the bases of the nucleotide residues. The complex may comprise two strands forming a duplex structure, three or more strands forming a multi stranded complex, a single self-hybridizing strand, or any combination of these. A sequence capable of hybridizing with a given sequence is referred to as the “complement” of the given sequence.

The synonymous terms “hydroxyl protecting group” and “alcohol-protecting group” as used herein refer to substituents attached to the oxygen of an alcohol group commonly employed to block or protect the alcohol functionality while reacting other functional groups on the compound. Examples of such alcohol-protecting groups include but are not limited to the 2-tetrahydropyranyl group, 2-(bisacetoxyethoxy)methyl group, trityl group, trichloroacetyl group, carbonate-type blocking groups such as benzyloxycarbonyl, trialkylsilyl groups, examples of such being trimethylsilyl, tert-butyldimethylsilyl, tert-butyldiphenylsilyl, phenyldimethylsilyl, triiospropylsilyl, triisopropylsilyloxymethyl (TOM) and thexyldimethylsilyl, ester groups, examples of such being formyl, (C1-C10) alkanoyl optionally mono-, di- or tri-substituted with (C1-C6) alkyl, (C1-C6) alkoxy, halo, aryl, aryloxy or haloaryloxy, the aroyl group including optionally mono-, di- or tri-substituted on the ring carbons with halo, (C1-C6) alkyl, (C1-C6) alkoxy wherein aryl is phenyl, 2-furyl, carbonates, sulfonates, and ethers such as benzyl, p-methoxybenzyl, methoxymethyl, 2-ethoxyethyl group, etc. The choice of alcohol-protecting group employed is not critical so long as the derivatized alcohol group is stable to the conditions of subsequent reaction(s) on other positions of the compound of the formula and can be removed at the desired point without disrupting the remainder of the molecule. Further examples of groups referred to by the above terms are described by J. W. Barton, “Protective Groups In Organic Chemistry”, J. G. W. McOmie, Ed., Plenum Press, New York, N.Y., 1973, and G. M. Wuts, T. W. Greene, “Protective Groups in Organic Synthesis”, John Wiley & Sons Inc., Hoboken, New Jersey, 2007, which are hereby incorporated by reference. The related terms “protected hydroxyl” or “protected alcohol” define a hydroxyl group substituted with a hydroxyl protecting group as discussed above.

The term “nitrogen protecting group,” as used herein, refers to groups known in the art that are readily introduced on to and removed from a nitrogen atom. Examples of nitrogen protecting groups include but are not limited to acetyl (Ac), trifluoroacetyl (TFA), isopropyl-phenoxyacetyl or phenoxyacetyl (PAC), Boc, Cbz, benzoyl (Bz), Fluorenylmethyloxycarbonyl (Fmoc), N,N-dimethylformamidine (DMF), trityl, Monomethoxytrityl (MMT), Dimethoxytrityl (DMTr), and benzyl (Bn). See also G. M. Wuts, T. W. Greene, “Protective Groups in Organic Synthesis”, John Wiley & Sons Inc., Hoboken, New Jersey, 2007, and related publications.

The term of “Isotopically enriched” refers to a compound containing at least one atom having an isotopic composition other than the natural isotopic composition of that atom. The term of “Isotopic composition” refers to the amount of each isotope present for a given atom, and “natural isotopic composition” refers to the naturally occurring isotopic composition or abundance for a given atom. As used herein, an isotopically enriched compound optionally contains deuterium, carbon-13, nitrogen-15, and/or oxygen-18 at amounts other than their natural isotopic compositions.

As used herein, the terms “therapeutic agent” and “therapeutic agents” refer to any agent(s) which can be used in the treatment or prevention of a disorder or one or more symptoms thereof. In certain embodiments, the term “therapeutic agent” includes a compound provided herein. In certain embodiments, a therapeutic agent is an agent known to be useful for, or which has been or is currently being used for the treatment or prevention of a disorder or one or more symptoms thereof.

Segmented Nucleic Acids

The invention relates, in part, to synthesis of segmented nucleic acids.

One embodiment is the synthesis of a three segmented single molecule guide RNA of CRISPR-Cas9, comprising:

1. Synthesis of 3′-amino two segmented RNA (SEQ ID NO: 3) comprising a crgRNA and tracrg1RNA (SEQ ID NO: 2) joined together by a triazole non-nucleotide linker;

2. Transformation of 3′-amino modifier to 3′-azido by a diazotransfer reaction with fluorosulfuryl azide to give 3′-azido crgRNA-tracrg1RNA (SEQ ID NO: 4);

3. Formation of a three segmented RNA (l2gRNA, SEQ ID NO: 6) by SPAAC reaction between 3′-azido crgRNA-tracrg1RNA (SEQ ID NO: 4) and 5′-alkynyl tracrg2RNA (SEQ ID NO: 5).

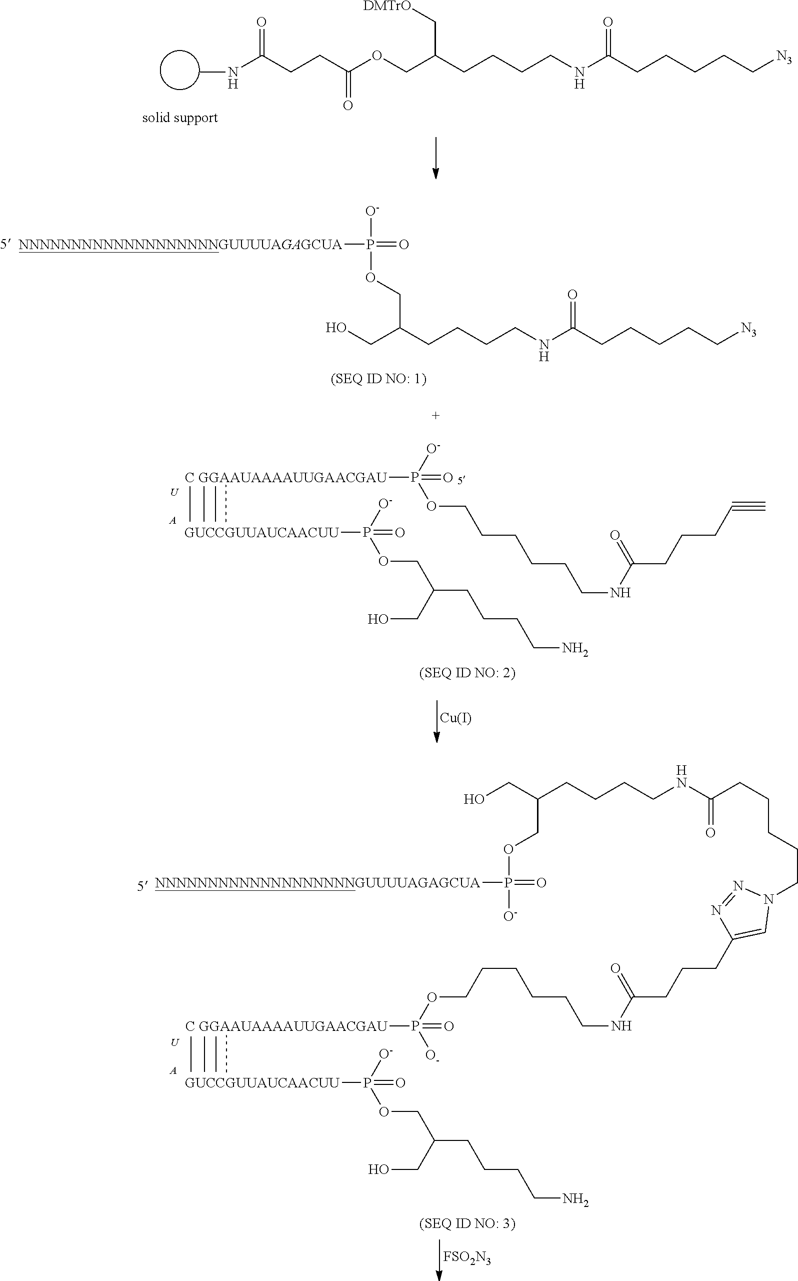

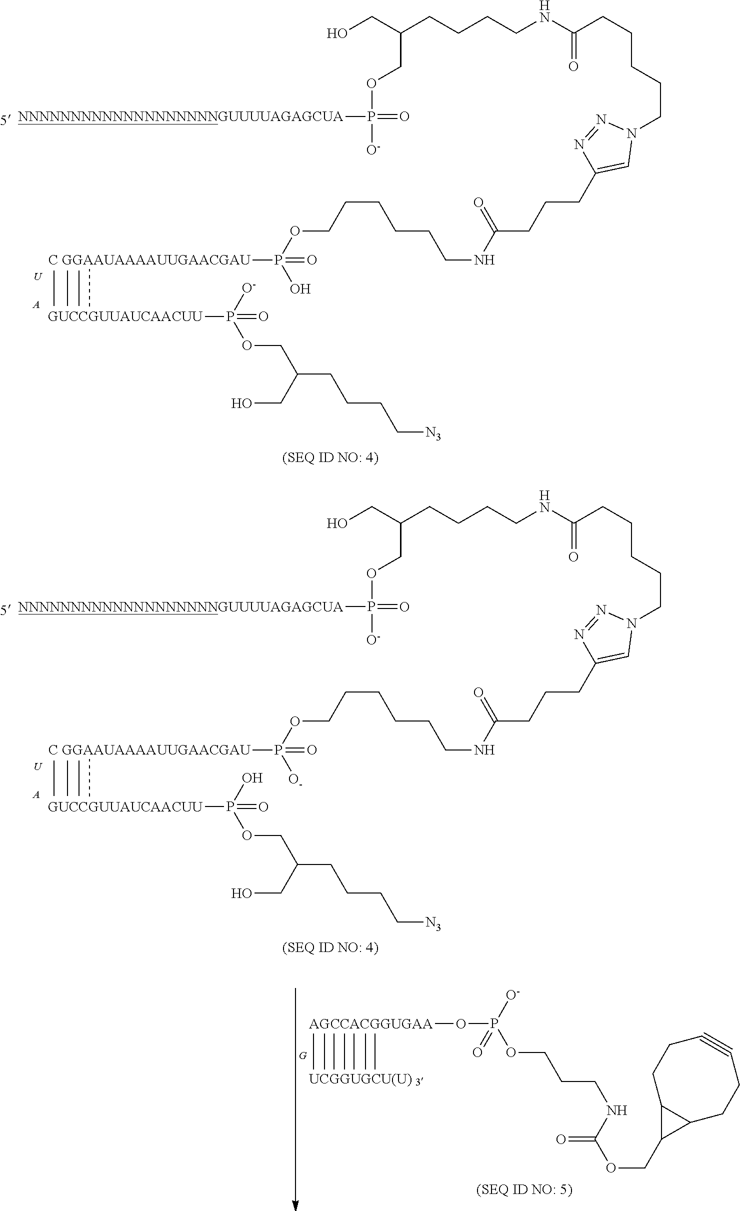

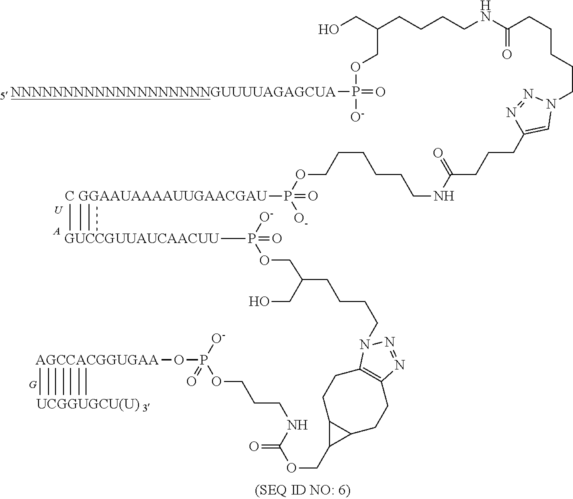

In one embodiment, formation of a three segmented RNA (l2gRNA) in step 3 is a CuAAC reaction between 3′-azido crgRNA-tracrg1RNA (SEQ ID NO: 4) and 5′-alkynyl tracrg2RNA (SEQ ID NO: 7).

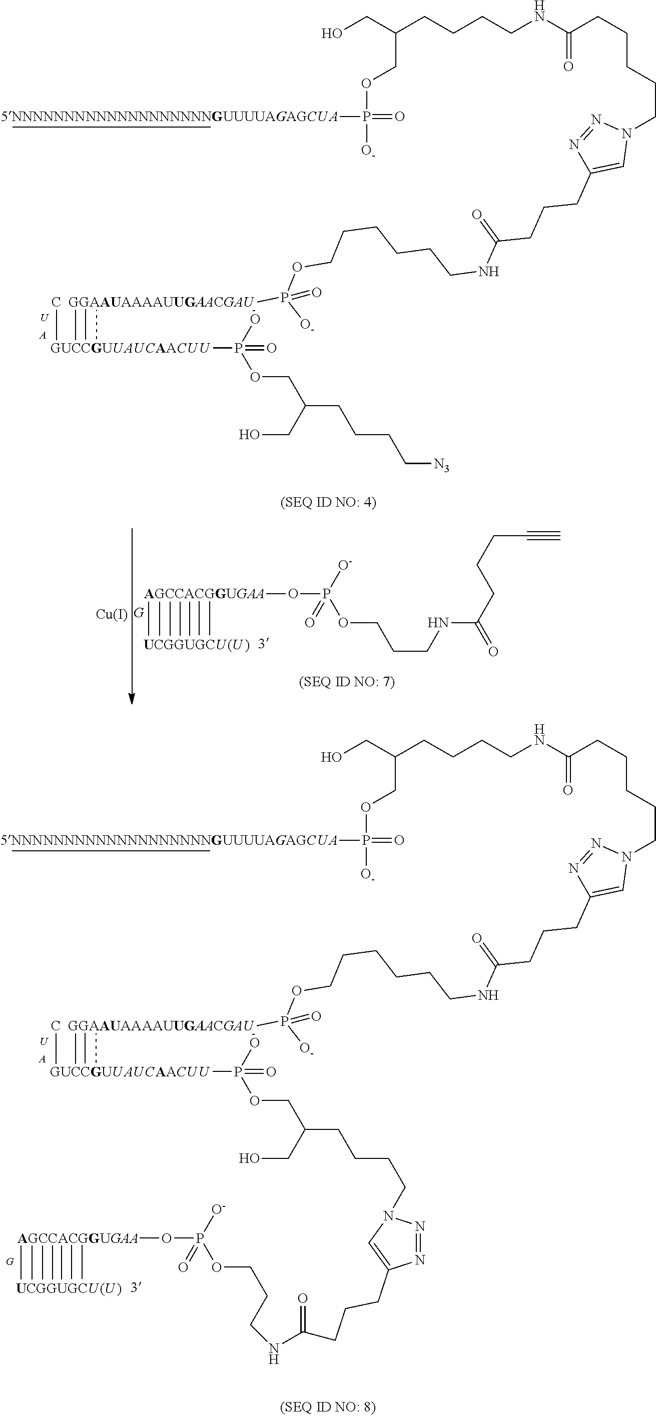

In another embodiment, formation of a three segmented RNA (l2gRNA) in step 3 is a Staudinger reaction between 3′-azido crgRNA-tracrg1RNA (SEQ ID NO: 4) and a phosphine (tracrg2RNA, SEQ ID NO: 9).

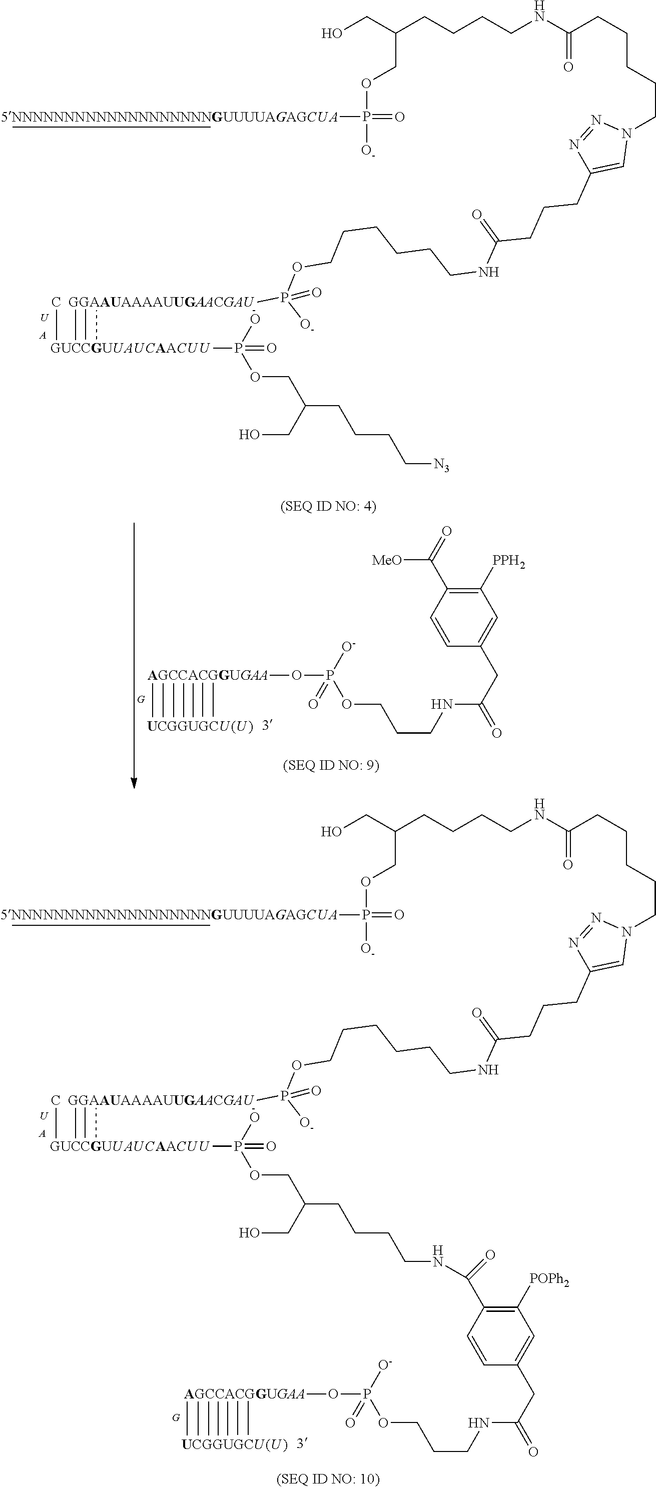

The invention relates, in part, to synthesis of conjugates of segmented nucleic acids.

One embodiment of the invention is synthesis of conjugates of l2gRNA (SEQ ID NO: 13) from 3′-amino l2gRNA (SEQ ID NO: 12).

In another embodiment, the conjugation site is either at the 5′-end, 3′-end, both, or non-nucleotide linker(s) of an l2gRNA.

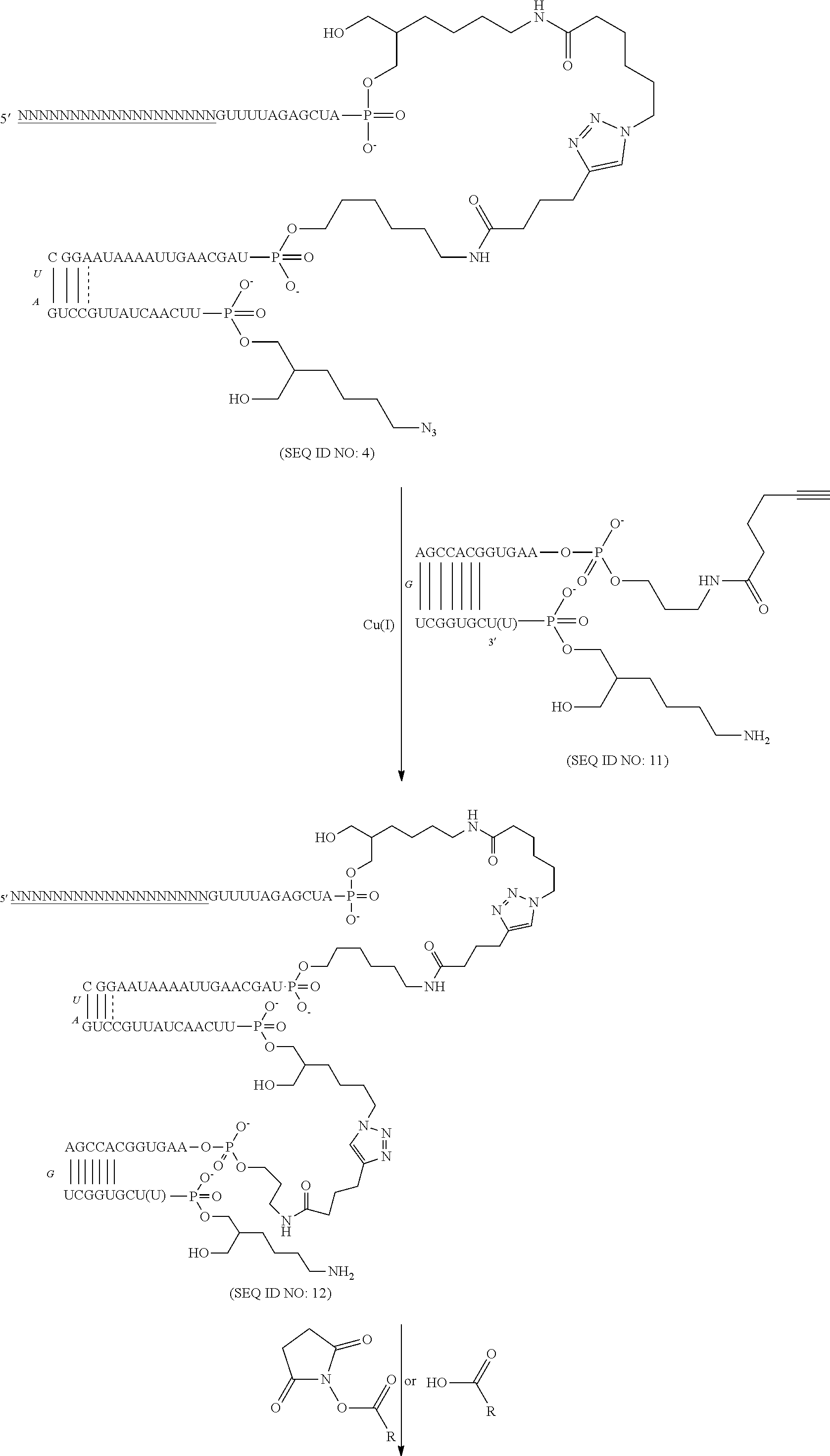

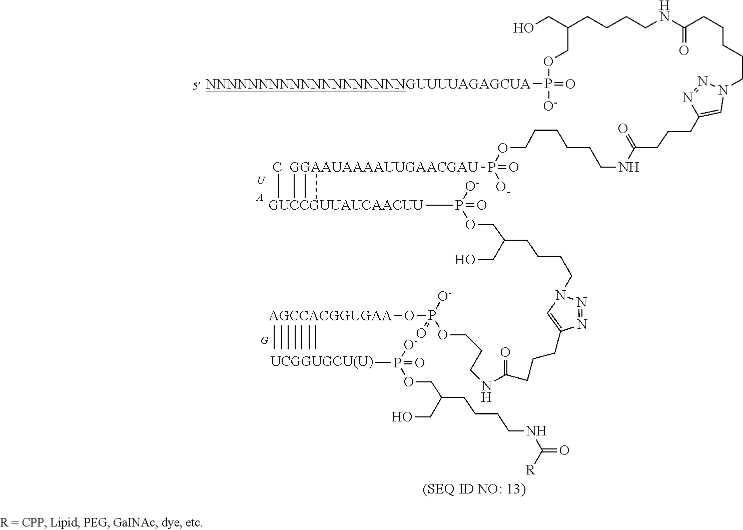

In one embodiment, the above tracrg2RNA (SEQ ID NO: 5, 7 and 9) is extended at 3′-end by an RNA template with edit for reverse transcription and a primer binding site. The l2gRNA products are chemically ligated three-segmented pegRNAs to be used in prime editing (See, e.g., Anzalone et al. Nature 2019, 576, 149-157.).

In one embodiment, an l2gRNA is synthesized from three segments as follows: 1. a ligated tracrgRNA (5′-amino 1-tracrRNA) is synthesized from two segments of tracrRNA (tracrgRNA1 and tracrgRNA2, SEQ ID NO: 15 and SEQ ID NO: 16, respectively.); 2. synthesis of an l2gRNA by click reaction of tracrgRNA with crgRNA (SEQ ID NO: 14) by a second click reaction after 5′-amnio 1-tracrRNA is transformed into 5′-azido 1-tracrRNA either by a diazotransfer reaction or an amide formation with an azido NHS ester.

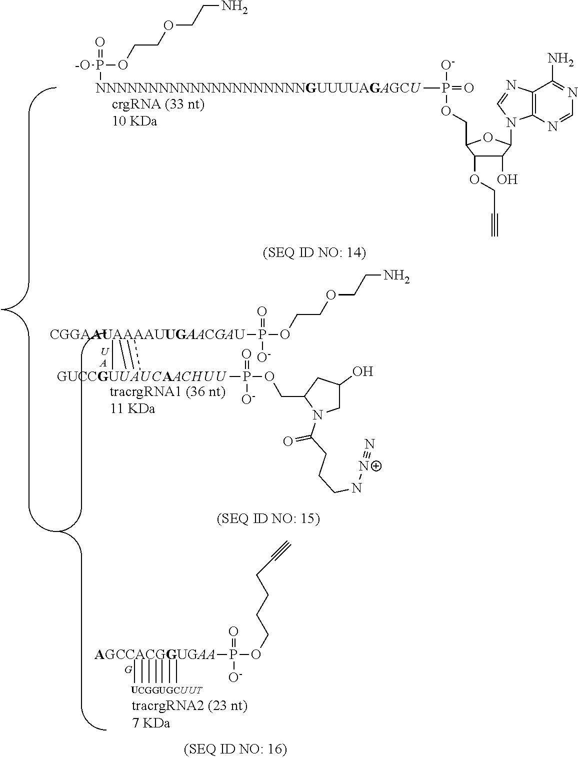

Synthesis of l2gRNA targeting eGFP as an example is given below.

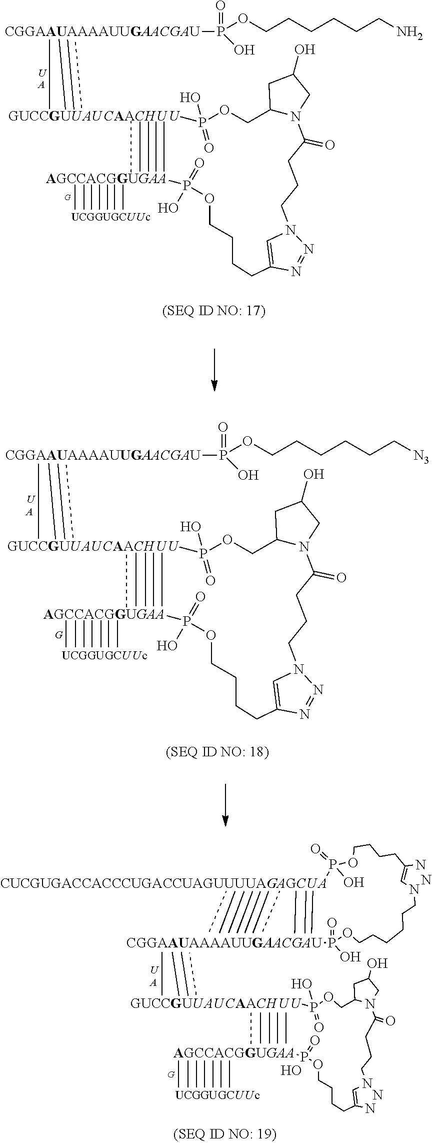

The invention further relates, in part, to synthesis of libraries of l2gRNA or their conjugates.

5′-Azido 1-tracrRNA (SEQ ID NO: 18) is synthesized at large scale (>1 mmole) and is ligated to a library of 3′-alkynyl or 3′-phospino crgRNAs with different spacers (target sequence) either in an arrayed form or a pooled form.

The invention still further relates, in part, to synthesis of l2gRNA-ssDNA templates (segRNA) and their conjugates used in a STAR editor (See, e.g., Zhong WO2021034373, the entire disclosure of which is incorporated herein by reference.).

One embodiment is synthesis of segRNA comprising a long dsDNA template for gene editing comprising the following steps: a. synthesis of three segment l2gRNA comprising a 3′-terminal adaptor ssDNA of ˜18-100 nt in length; b. annealing with a complementary ssDNA to form a dsDNA; c. the formed dsDNA in step b is processed by a DNA endonuclease to form a sticky 3′ end; d. connecting the 5′-end of a dsDNA template processed by an appropriate endonuclease to the 3′ end of the product in step c by a DNA ligase.

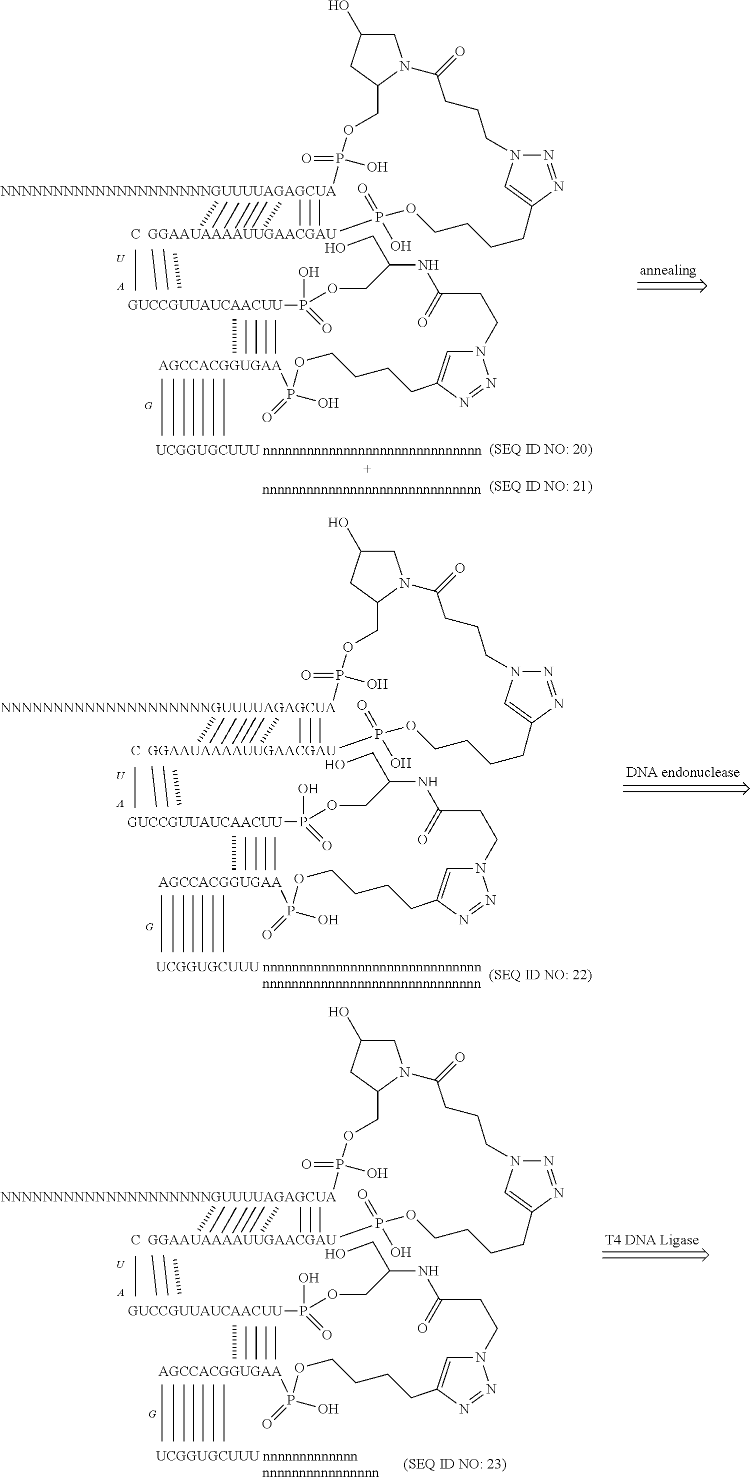

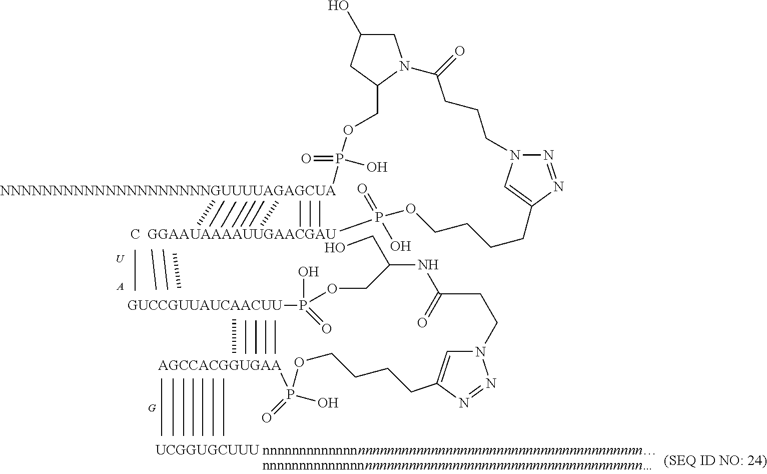

Another embodiment is synthesis of a segRNA comprising an ssDNA template formed by hybridization between the adaptor DNA sequence (covalently linked to 3′-end of l2gRNA) of ˜18-100 nt in length and 5′-end of the ssDNA template (SEQ ID NO: 25).

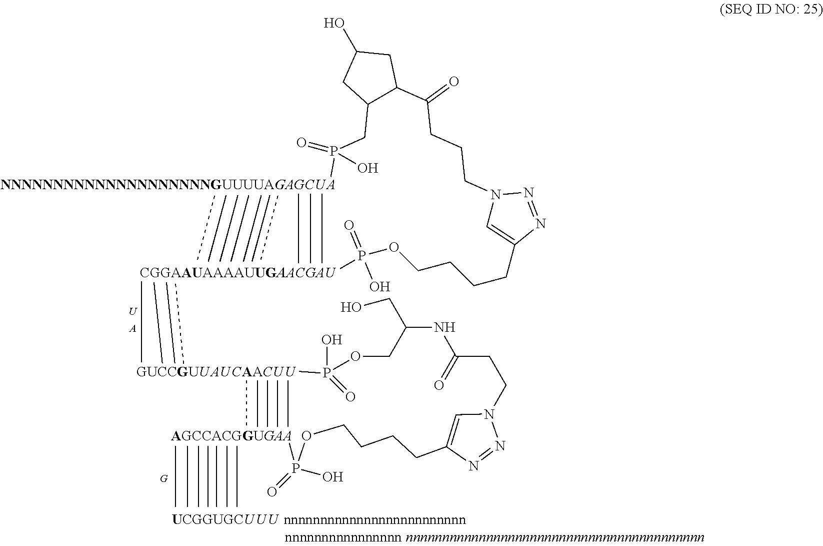

Another embodiment is synthesis of guide RNA, comprising a specificity domain of ˜18-100 nt in length and a ligated ADAR recruiting domain, to complex with an endogenous human ADAR enzyme (SEQ ID NO: 27).

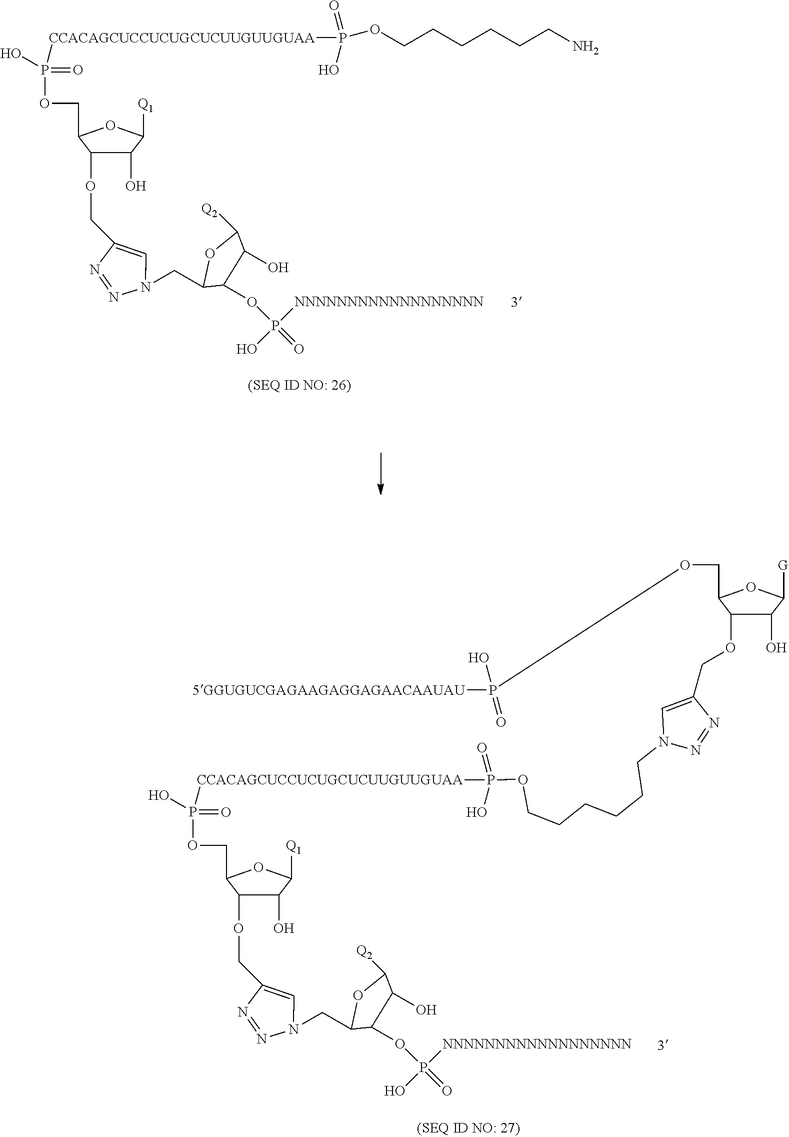

Another embodiment is syntheses of guide RNA conjugates via 5′- or 3′-amino segmented guide RNAs for cellular delivery in ADAR mediated RNA editing.

Another embodiment is syntheses of guide RNA conjugates comprising two-segment nucleic acid (a specificity domain of ˜15-100 nt and an ssRNA of ˜15-40 nt, which forms an ADAR recruiting domain with a target mRNA, joined by a non-nucleotide linker) and 5′- and/or 3′-conjugated chemical moieties for selective cellular delivery.

RNA Guided Gene Editing Proteins

The invention relates, in part, to segmented-RNA guided gene editing enzymes, including CRISPR Cas mediated editing of nucleic acids and ADAR mediated RNA editing.

CRISPR endonucleases and fusion proteins. In some embodiments, CRISPR effector endonuclease is selected from Cas proteins of Type II, Class 2 including Streptococcus pyogenes-derived Cas9 (SpCas9, 4.1 kb), smaller Cas9 orthologues, including Staphylococcus aureus-derived Cas9 (SaCas9, 3.16 kb), Campylobacter jejuni-derived Cas9 (CjCas9, 2.95 kb), Streptococcus thermophilus Cas9 (StlCas9, 3.3 kb), Neisseria meningitidis (NmCas9, 3.2 kb), and other variants of engineered Cas9 proteins such as SpCas9-HFl, eSpCas9, and HypaCas9, proteins of Type V, Class 2 including Cas12 (Cas12a (Cpfl), Cas12b (C2c1), Cas 12c, Cas12e,Cas12g, Cas12h, Cas12i, and etc.) and Cas14, and proteins of Type VI, Class 2 such as Cas13a and Cas 13b. The said CRISPR effector protein can be a nickase, e.g., SpCas9-nickase (D10A or H840A), or a catalytically inactive protein, e.g., Cas9 (dCas9) coupled/fused with a protein effector such as Fokl, transcription activator(s), transcription repressor(s), catalytic domains of DNA methyltransferase, histone acetyltransferase and deacetylase, reverse transcriptase (prime editor), integrase, ligase, and nucleic acid deaminases (base editor).

In another embodiment, the said CRISPR effector endonuclease is an artificial one comprising one or more functional domains derived from human.

ADAR enzymes. ADAR (encoded by ADAR, also known as ADAR1 or DSRAD) carries out adenosine-to-inosine (A-to-I) editing within double-stranded RNA (dsRNA). Three members of this protein family, ADAR1-3, are known to exist in mammalian cells. ADAR3 is a catalytically null enzyme and the most significant function of ADAR2 was found to be in editing on the neuron receptor GluR-B mRNA. ADAR1, however, has been shown to play more significant roles in biological and pathological conditions.

One embodiment of the invention is the syntheses and uses of segmented guide RNAs and their conjugates to complex with endogenous ADAR enzyme(s) for treatment of diseases.

Non-Nucleotide Linkers for Active Secondary Structures of Guide RNAS

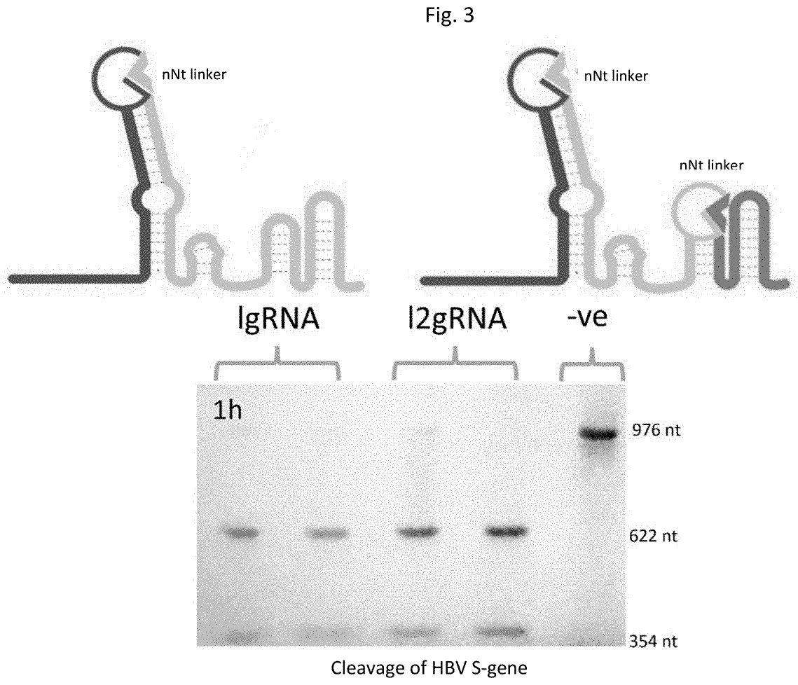

In one embodiment, one or more non-nucleotide linkers are positioned in a segmented Cas9 guide RNA for locking the RNA to active secondary structure, i.e., l2gRNA in FIG. 3, and preventing RNA misfolding.

In one embodiment, non-nucleotide linker(s) positioned at GAAA tetraloop(s) has two cis-side chains covalently bonded to a locked structure such as a substituted proline, e.g., with the amine and the carboxylic acid function, respectively, and locks the hairpin structure.

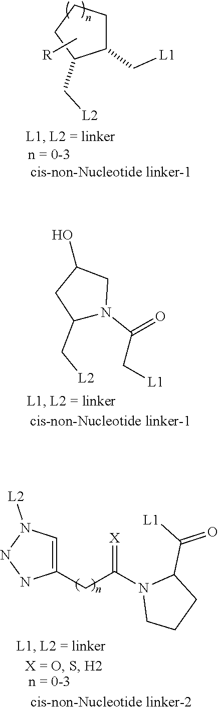

In another embodiment, a non-nucleotide linker positioned at a loop exposed out of the bound region of the guide RNA comprises a chemical moiety capable of positioning the two side chains of the non-nucleotide linker into a cis configuration.

In yet another embodiment, a non-nucleotide linker positioned at a stem of the guide RNA comprises a chemical moiety capable of positioning the two side chains of the non-nucleotide linker into a locked linear configuration.

### EXAMPLES

The following examples further illustrate embodiments of the disclosed invention, which are not limited by these examples.

### Example 1

### Compound 1

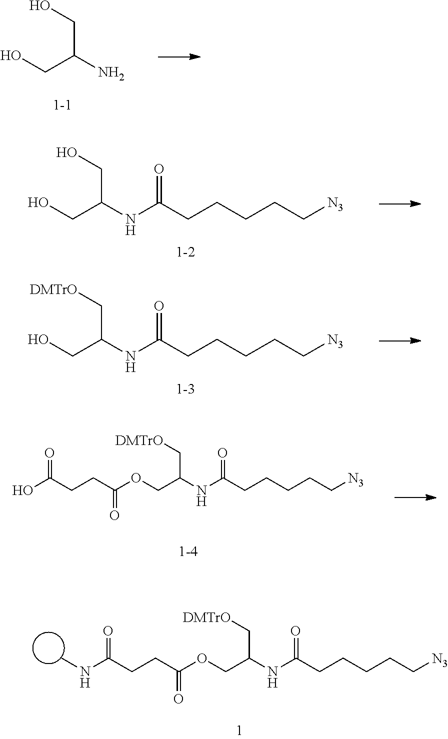

Serinol (456 mg, 50 mmole) is treated with 6-azido hexanoic acid (786 mg, 50 mmole), EDCI (1.06 g, 55 mmole), and NHS (633 mg, 55 mmole) in dimethylformamide (15 mL), and the resulting mixture is stirred at room temperature overnight. The mixture is concentrated under vacuum, and the residue is separated by a flash column (MeOH/DCM, 0→10%) to give the immediate 1-2.

Compound 1-2 is tritylated (DMTrCl, in pyridine, RT), and attached to an amino-functionalized support to provide compound 1.

### Example 2

### crgRNA-eGFP

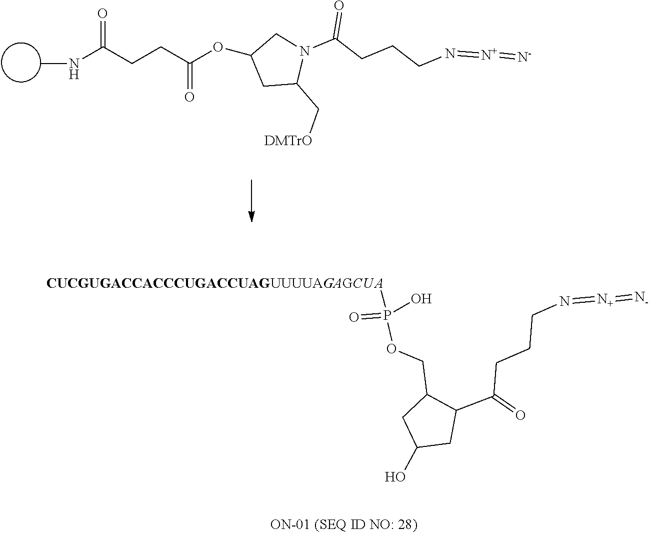

ON-01 was prepared on an Expedite 8909 automated DNA/RNA synthesizer using the standard 1.0 μmole RNA phosphoramidite cycle. 3′-Azido CPG 1000 Å (1 μmole) was packed into an Expedite column. All β-cyanoethyl phosphoramidite monomers were dissolved in anhydrous acetonitrile to a concentration of 0.1 M immediately prior to use. Coupling, capping and oxidation reagents (ChemGenes) were 5-Ethyl-1H-tetrazole (0.45 M in acetonitrile), Cap A (Acetic Anhydride/Pyridine/THF)/Cap B (10% N-Methylimidazole in THF) and iodine (0.02M Iodine/Pyridine/H₂O/THF), respectively. Stepwise coupling efficiencies were determined by automated trityl cation conductivity monitoring and in all cases were >97%.

Oligonucleotide on solid support was treated with 20% piperidine in DMF at room temperature to suppress the formation of cyanoethyl adducts, then washed with acetonitrile (3×1 mL) and dried with argon.

RNA deprotection. The oligonucleotide on solid support was exposed to AMA (Ammonium Hydroxide/40% aqueous Methylamine 1:1 v/v) in a sealed vial for 20 min at 65° C. The solution was collected by filtration and the solution was then concentrated till dryness in a Savant SpeedVac concentrator at room temperature. The resulting white solid was re-dissolved in a 2:2:3 v/v mixture of dry NMP (200 μL), triethylamine (200 μL) and triethylamine trihydrofluoride (300 μL) and heated at 60° C. for 3 h. After cooling down to room temperature, sodium acetate (3M pH 5.2, 40 μL) and ethanol (1 mL) were added and the RNA was stored for 30 min at −78° C. The RNA was then pelleted by centrifugation (15,850×g, 10 min, 4° C.), the supernatant discarded and the pellet washed twice with 70% ethanol (500 μL). The pellet was then dried in vacuo and used for next step without further purification.

### Example 3

### tracrgRNA-eGFP

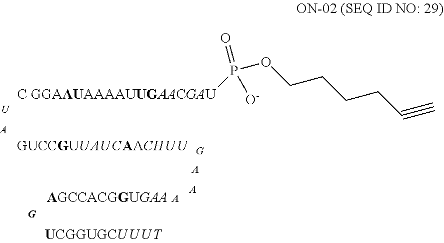

ON-02 was prepared on an Expedite 8909 automated DNA/RNA synthesizer using the standard 1.0 μmole RNA phosphoramidite cycle, fully deprotected and separated as ON-01. Thymidine 3′-lcaa CPG 1000 Å (1 μmole) was used instead. The pellet was then dried in vacuo and used for next step without further purification.

### Example 4

### lgRNA-eGFP

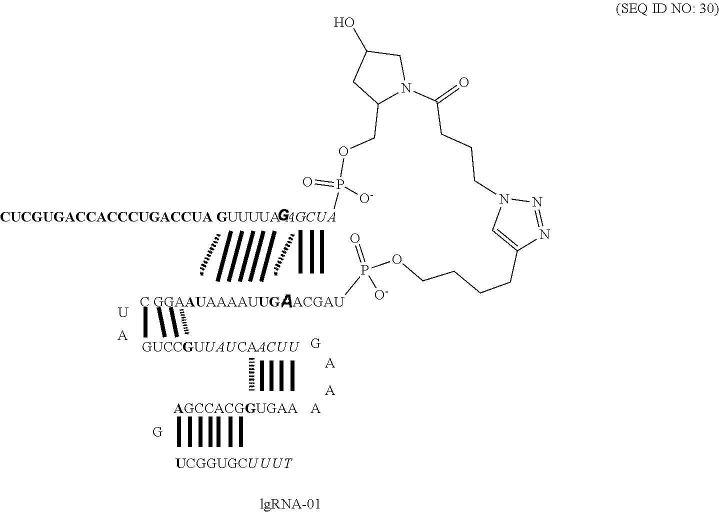

To azide ON-1 pellet (half, <0.49 μmole) and alkyne ON-2 pellet (half, <0.49 μmole) in a stock solution (DMSO/ddH₂O/2 M TEAA, 2:1:0.4, 1700 μL) was added CuSO₄-THPTA (tris-hydroxypropyl triazole ligand) (250 mM, 100 μL), and the resulting light blue solution was deoxygenated by bubbling argon for 10 min. Freshly prepared ascorbic acid in ddH₂O (125 mM, 200 μL) was added, and reaction mixture was further deoxygenated by bubbling argon for 30 min. The reaction mixture was sealed and kept at room temperature for 2 h, and sodium acetate (3 M pH 5.2, 40 μL) and ethanol (1 mL) were added. The resulting RNA suspension was stored for 30 min at −78° C. The RNA was then pelleted by centrifugation (15,850×g, 10 min, 4° C.). The supernatant was discarded and the pellet washed twice with 70% ethanol (500 μL). The pellet was then dried in vacuo at room temperature.

The above oligonucleotide pellet was mixed with gel loading buffer (formamide/ddH₂O 90% v/v, with 10 mM EDTA) and RNA loading dyes (2×) and loaded onto a denaturing 10% polyacrylamide gel (1×TBE buffer containing 7M urea) and separated at 65W for 2-3 h. RNA bands were visualized under UV, excised, crushed, soaked in a gel extract buffer (NaCl solution with 2 mM EDTA) overnight at 37° C. with vigorous shaking. The gel was removed by filtration through two consecutive Sep-Pak C18 plus short cartridges, the oligonucleotide solutions were combined, and the final concentration was determined by a NanoDrop spectrophotometer at 260 nm. The solution was concentrated till dryness in vacuo in a Savant SpeedVac concentrator at room temperature.

The product (lgRNA-01) was analyzed by ESI-LCMS (Novatia, LLC). Calculated mass: 31137 Da; observed mass: 31143 Da.

### Example 5

### ON-04

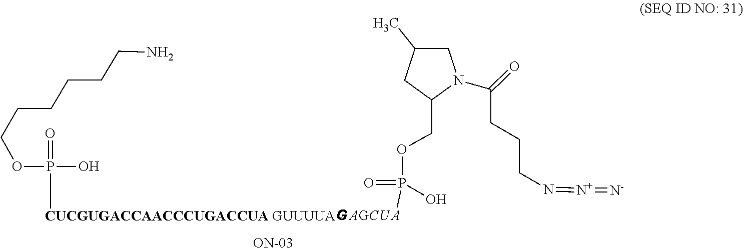

ON-03 was synthesized and separated as ON-01. The 5′-amino modifier was introduced with TFA-amino C-6 CED phosphoramidite. The oligonucleotide on solid support was treated with 20% piperidine in DMF at room temperature to suppress the formation of cyanoethyl adducts, was then washed with acetonitrile (3×1 mL) and dried with argon.

Cleavage of oligonucleotides from the solid support and deprotection were achieved by exposure to AMA at 65° C. for 20 min, followed by desilylation and ethanol precipitation as before. The pellet was then dried in vacuo and used for next step without further purification.

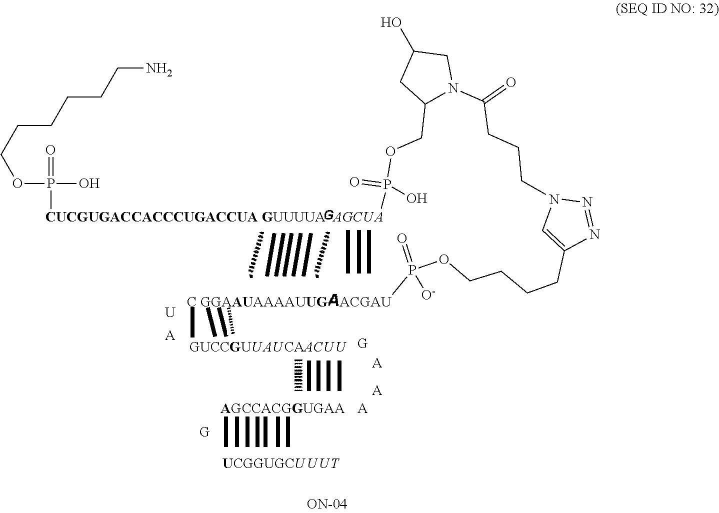

The product (ON-04) was prepared by CuAAC ligation between the two pellets (ON-02 and ON-03) as above, and analyzed by ESI-LCMS (Novatia, LLC). Calculated mass: 31,317 Da; observed mass: 31,319 Da.

### Example 5

### ON-05

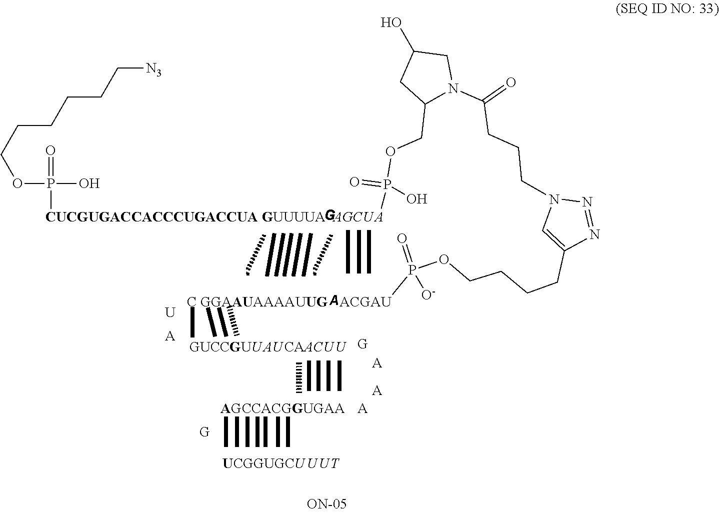

The 5′-amino ON-04 was transformed to 5′-azido ON-05 by a diazotransfer reaction. ON-04 (25 nmoles) was dissolved in 0.1 M NaHCO₃, pH 8.5 (300 μL) and DMF (60 μL), and FSO₂N₃ in MTBE (˜0.5 M, 300 μL) was added. The mixture was thoroughly mixed for 30 min at room temperature, and then kept at rest for 30 min. The reaction mixture was centrifuged at 15,000 rpm for 10 min, and organic and aqueous layers were well separated. The colorless organic phase was removed from residual aqueous phase containing the oligonucleotide. To the aqueous phase were added 3 M NaOAc (40 μL) and ethanol (1000 μL). The RNA suspension was stored for 30 min at −78° C. The RNA was then pelleted by centrifugation (15,850×g, 10 min, 4° C.). The supernatant discarded and the pellet washed twice with 70% ethanol (500 μL). The pellet was then dried in vacuo at room temperature.

### Example 6

### segRNA-eGFP-01

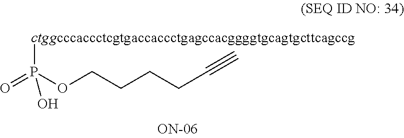

ON-06 was prepared on an Expedite 8909 automated DNA/RNA synthesizer using the standard 1.0 μmole DNA phosphoramidite cycle. dG 3′-lcaa CPG 1000 Å (1 μmole) was packed into an Expedite column. All β-cyanoethyl phosphoramidite monomers were dissolved in anhydrous acetonitrile to a concentration of 0.1 M immediately prior to use. Coupling, capping and oxidation reagents (ChemGenes) were 1H-tetrazole (0.5 M in acetonitrile), Cap A (Acetic Anhydride/Pyridine/THF)/Cap B (10% N-Methylimidazole in THF) and iodine (0.02 M Iodine/Pyridine/H₂O/THF), respectively. Stepwise coupling efficiencies were determined by automated trityl cation conductivity monitoring and in all cases were >99%.

The oligonucleotide on solid support was exposed to (Ammonium Hydroxide/ethanol 3:1 v/v) in a sealed vial for 10 h at 55° C. The solution was collected by filtration and concentrated till dryness in a Savant SpeedVac concentrator at room temperature. To the resulting white solid, ddH₂O (100 μL), sodium acetate (3 M pH 5.2, 40 μL) and ethanol (1 mL) were added sequentially and the DNA suspension was stored for 30 min at −78° C. The DNA was then pelleted by centrifugation (15,850×g, 10 min, 4° C.), the supernatant discarded and the pellet washed twice with 70% ethanol (500 μL). The pellet was then dried in vacuo and used for next step without further purification.

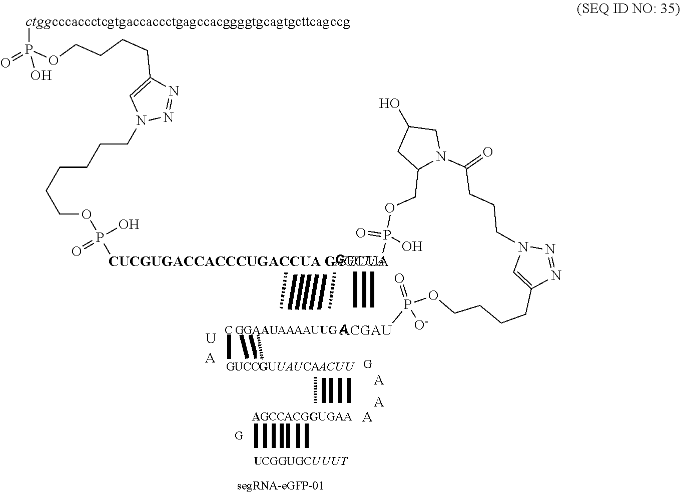

ON-05 and ON-06 were ligated by CuAAC reaction as above, and the resulting product was separated by ethanol precipitation. The resulting pellet was dried under vacuum, and separated by denaturing PAGE to give segRNA-eGFP-01. Calculated mass: 47078 Da; observed mass: 47079 Da.

### Example 7

### segRNA-eGFP-02

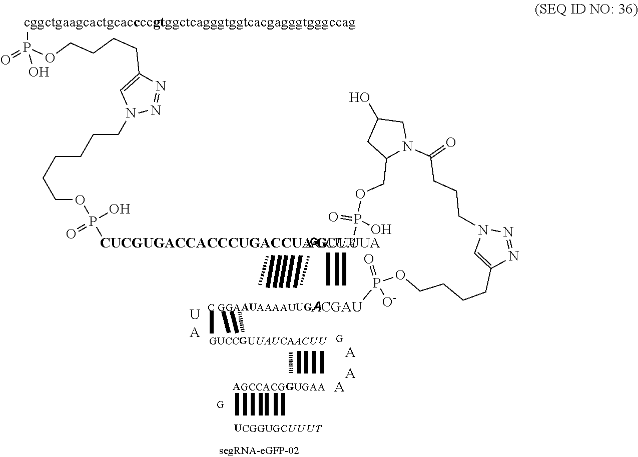

SegRNA-eGFP-02 was prepared as segRNA-eGFP-01. Calculated mass: 47327 Da; observed mass: 47330 Da.

### Example 8

### ON-07

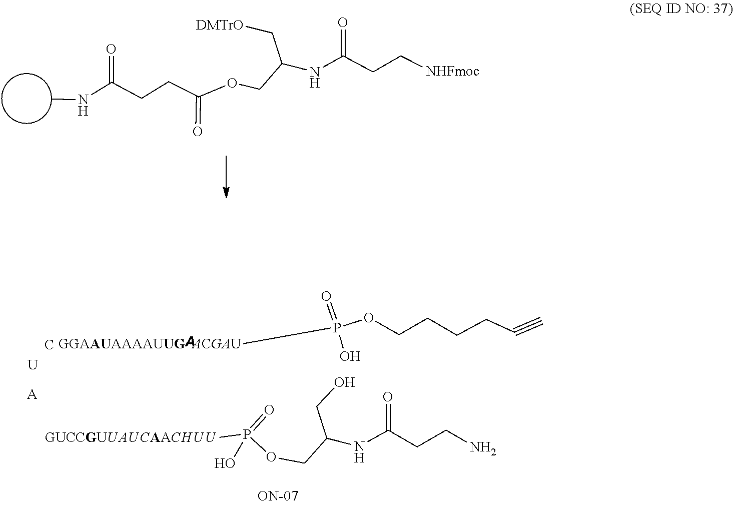

ON-07 was prepared on an Expedite 8909 automated DNA/RNA synthesizer using the standard 1.0 μmole RNA phosphoramidite cycle. 3′-Amino modified serinol CPG 1000 A (1 μmole) was used instead.

Oligonucleotide on the solid support was treated with 20% piperidine in DMF at room temperature to remove the Fmoc protection, was then washed with acetonitrile (3×1 mL) and dried with argon.

The RNA was then fully deprotected as ON-01. The resulting pellet was dried in vacuo and used for next step without further purification.

### Example 4

### ON-08

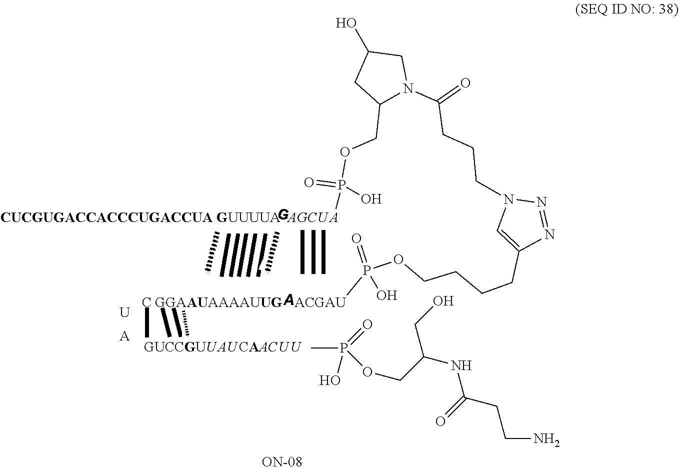

ON-07 and ON-01 were ligated by CuAAC reaction as above, and the resulting product was separated by ethanol precipitation. The resulting pellet was dried under vacuum, and separated by denaturing PAGE to give ON-08.

The 3′-amino ON-08 was transformed to 3′-azido ON-09 by a diazotransfer reaction with FSO₂N₃ in a way similar to ON-05.

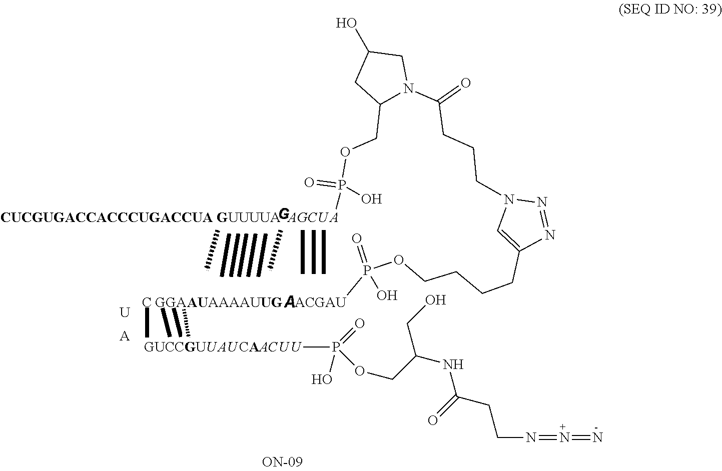

Alternatively, ON-08 is dissolved in 0.5 M Na₂CO₃/NaHCO₃ buffer (pH 8.5) and incubated with 4-Azidobutyrate NHS ester (20 eq.) in DMSO to give ON-10.

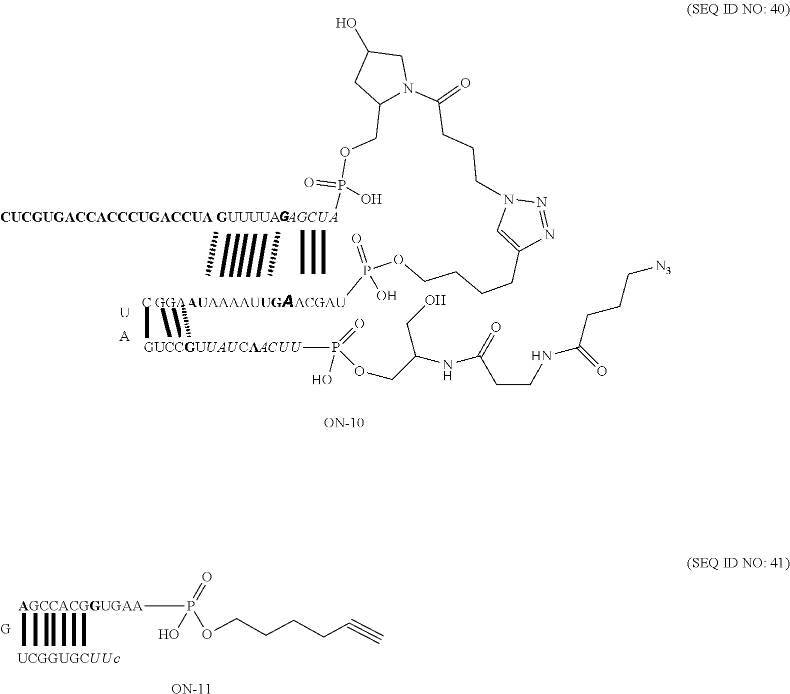

ON-11 was synthesized in a way similar to the synthesis of ON-02. dC 3′-lcaa CPG 1000 Å (1 μmole) was used instead. The pellet was then dried in vacuo and used for next step without further purification.

### Example 5

### l2gRNA-eGFP

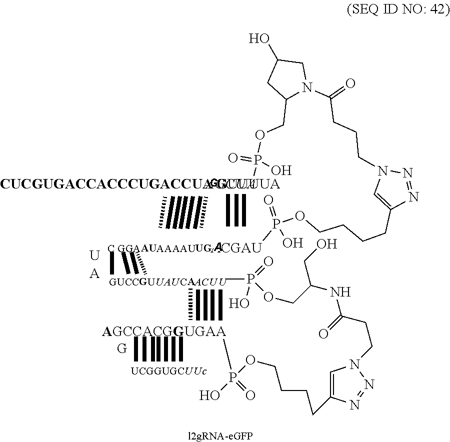

The product (l2gRNA-eGFP) was prepared by CuAAC ligation between the two pellets (ON-09 and ON-11) as above, and analyzed by ESI-LCMS (Novatia, LLC). Calculated mass: 29,832 Da; observed mass: 29,833 Da.

Alternatively, CuAAC ligation between ON-10 and ON-11 gives l2gRNA-eGFP with a non-nucleotide linker of increased length.

### Example 5

### ON-12

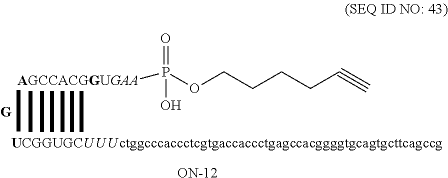

ON-12 is synthesized in a way similar to the synthesis of ON-02. dG 3′-lcaa CPG 1000 Å (1 μmole) was used instead. 1H-tetrazole (0.5 M in acetonitrile) was used as the activator for the DNA segment, while 5-Ethyl-1H-tetrazole (0.45 M in acetonitrile) for the RNA segment. The oligonucleotide on the solid support was deprotected and separated as ON-01. The pellet was then dried in vacuo and used for next step without further purification.

### Example 6

### segRNA-eGFP-03

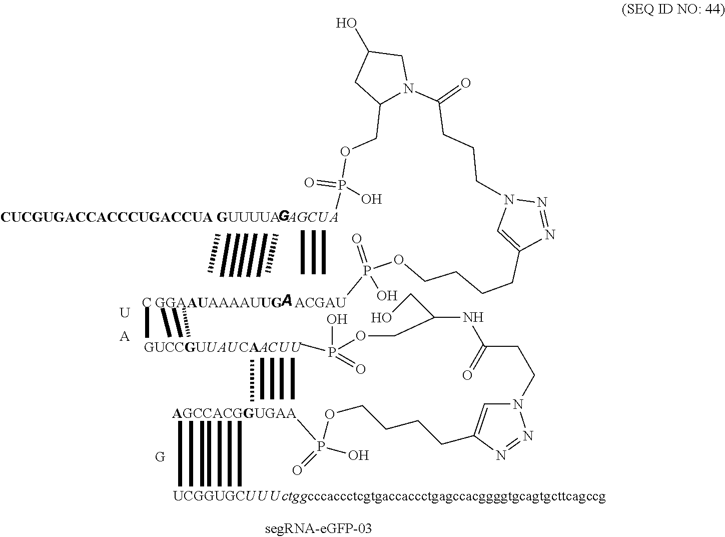

The product (segRNA-eGFP-03) was prepared by CuAAC ligation between the two pellets (ON-09 and ON-12) as above, and analyzed by ESI-LCMS (Novatia, LLC). Calculated mass: 45486 Da; observed mass: 45487 Da.

### Example 7

### segRNA-eGFP-04

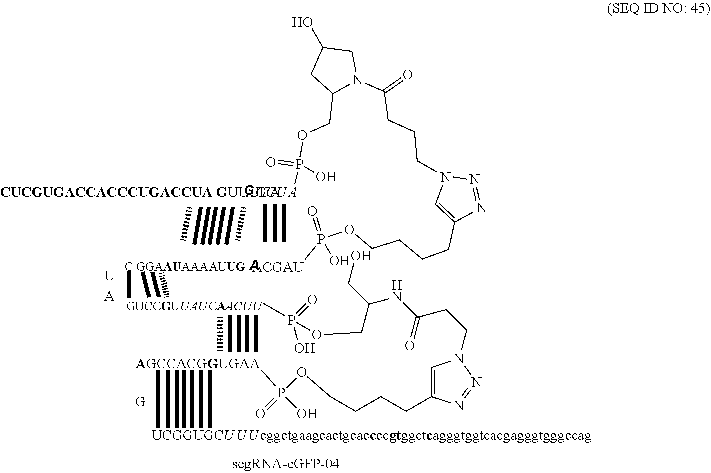

The product (segRNA-eGFP-04) was prepared by CuAAC ligation as above, and analyzed by ESI-LCMS (Novatia, LLC). Calculated mass: 45735 Da; observed mass: 45736 Da.

### Example 7

### In Vitro Cleavage Assay

Recombinant Cas9 protein was purchased from New England BioLabs, Inc. Cas9 and lgRNA or segRNA were preincubated in a 1:1 molar ratio in the cleavage buffer to reconstitute the RNP complex.

The substrate of HBV S gene (type ayw) or a dsDNA comprising eGFP and partial HBV S gene was dissolved in the cleavage buffer and added to the RNP complex. The reaction mixture was incubated at 37° C. for 1 h, and DNA loading dyes (6×) was added. The resulting mixture was heated at 95° C. for 5 min, cooled to room temperature, and resolved by a 1% Agarose gel.

### Example 8

### In Vitro Gene Editing

293/GFP cells (Cell Biolabs) are passaged on the day prior to electroporation.

100 pmol of Cas9-2NLS (or variants) is diluted to a final volume of 5 μL with Cas9 buffer (20 mM HEPES (pH7.5), 150 mM KCl, 1 mM MgCl₂, 10% glycerol and 1 mM TCEP) and mixed slowly into 5 μL of Cas9 buffer containing 120 pmol of lgRNA or segRNA. The resulting mixture is incubated for 10 min at room temperature to allow RNP formation. 2×10⁻⁵ 293/GFP cells are harvested, washed once in PBS, and resuspended in 20 μL of SF nucleofection buffer (Lonza, Basel, Switzerland). 10 μL of RNP mixture and cell suspension are combined in a Lonza 4d strip nucleocuvette. Reaction mixtures are electroporated using setting DS150, incubated in the nucleocuvette at room temperature for 10 min, and transferred to culture dishes containing pre-warmed media. Editing outcomes are measured 4 and 7 days post-nucleofection by flow cytometry.

### Example 9

### Formation of Cas9-gRNA Complex, Cellular Transfections, and Assays

a. Transfection with cationic lipids (See, e.g., Liu et al. Nature Biotechnology 2015, 33, 73-80, the entire disclosure of which is incorporated herein by reference): Purified synthetic gRNA (lgRNA, l2gRNA or segRNA) or mixture of synthetic gRNAs is incubated with purified Cas9 protein for 5 min, and then complexed with the cationic lipid reagent in 25 μL OPTIMEM. The resulting mixture is applied to the cells for 4 h at 37° C.

b. Transfection with cell-penetrating peptides (See, e.g., Kim et al. Genome Res. 2014, 24: 1012-1019, the entire disclosure of which is incorporated herein by reference): Cell-penetrating peptide (CPP) is conjugated to a purified recombinant Cas9 protein (with appended Cys residue at the C terminus) by drop wise mixing of 1 mg Cas9 protein (2 mg/mL) with 50 μg 4-maleimidobutyryl-GGGRRRRRRRRRLLLL (m9R; 2 mg/mL) in PBS (pH 7.4) followed by incubation on a rotator at room temperature for 2 h. To remove unconjugated 9mR, the samples are dialyzed against DPBS (pH 7.4) at 4° C. for 24 h using 50 kDa molecular weight cutoff membranes. Cas9-m9R protein is collected from the dialysis membrane and the protein concentration is determined using the Bradford assay (Biorad).

Synthetic gRNA (lgRNA, l2gRNA or segRNA) or a mixture of synthetic gRNAs is complexed with CPP: gRNA (1 μg) in 1 μl of deionized water is gently added to the C3G9R4LC peptide (9R) in gRNA:peptide weight ratios that range from 1:2.5 to 1:40 in 100 μl of DPBS (pH 7.4). This mixture is incubated at room temperature for 30 min and diluted 10-fold using RNase-free deionized water.

150 μl Cas9-m9R (2 μM) protein is mixed with 100 μl gRNA:9R (10:50 μg) complex and the resulting mixture is applied to the cells for 4 h at 37° C. Cells can also be treated with Cas9-m9R and lgRNA:9R sequentially.

### Example 10

### In Vivo Gene Editing by LNP Mediated Delivery

LNP Formulations.

LNPs are prepared using a NanoAssemblr microfluidic system (Precision Nanosystems) as reported (See, e.g., Qiu et al. Proc Natl Acad Sci USA. 2021, 118(10):e2020401118, the entire disclosure of which is incorporated herein by reference.). Lipids (6Z,9Z,28Z,31Z)-heptatriaconta-6,9,28,31-tetraen-19-yl 4-(dimethylamino) butanoate (MC-3), DSPC, Cholesterol, and DMG-PEG2000 are dissolved in pure ethanol at a molar ratio of 50% MC-3, 38.5% Cholesterol, 10% DSPC, and 1.5% DMG-PEG2000 with a final MC-3 concentration of 10 mg/mL. Cas9 mRNA and gRNA (lgRNA, l2gRNA or segRNA) are mixed at the appropriate weight ratio in sodium acetate buffer (25 mM, pH 5.2). The RNA solution and the lipid solution are each injected into the NanoAssemblr microfluidic device at a ratio of 3:1, and the device results in the rapid mixing of the two components and thus the self-assembly of LNPs. Formulations are further dialyzed against PBS (10 mM, pH 7.4) in dialysis cassettes overnight at 4° C. The particle size of formulations is measured by dynamic light scattering (DLS) using a ZetaPALS DLS machine (Brookhaven Instruments). RNA encapsulation efficiency is characterized by Ribogreen assay.

In Vivo Gene Editing by LNP Delivery.

The above RNA-LNPs are intravenously injected into mice at a dose of 0.5 mg/kg RNA.

### Example 11

### Multiplexing Gene Editing

SegRNAs or segRNA conjugates are synthesized and mixed in an appropriate ratio. The mixture is either delivered with an mRNA or a plasmid or a viral vector encoding a CRISPR Cas protein, or complexes with a Cas protein or a Cas protein conjugate in vitro, and is delivered to target cells as a mixture of RNP complexes.

In case that the spacer and the ssDNA template/adaptor of a segRNA are covalently linked as a two-segmented nucleic acid, segRNAs or segRNA conjugates are alternatively synthesized as a mixture (pooled synthesis). ESI-LCMS is used to determine the ratio of each segRNA in the pool. The mixture is either delivered with an mRNA encoding a CRISPR Cas protein, or complexes with a Cas protein or a Cas protein conjugate in vitro, and is delivered to target cells as a mixture of RNP complexes.

For in vivo tests, the above mixtures, either alone or with additives such as transfection reagents, are intravenously injected into an animal.

### Example 12

### Anti-HBV in Cells

The antiviral assay is performed according to reported procedures (Yang et al. Molecular Therapy—Nucleic Acids, 2020, 20, 480-490; Lin et al. Molecular Therapy—Nucleic Acids, 2014, 3, e186, the entire disclosures of which are incorporated herein by reference.). Delivery to cell lines is either cationic lipid or CPP based delivery of Cas9-segRNA complexes instead of plasmid transfection/transduction using gRNA/Cas9 expression vectors.

Alternatively, cells are treated with segRNA and mRNA encoding Cas9 protein (segRNA/mRNA˜10:1) either as a mixture or sequentially in LNPs formulated with an amine-to-RNA-phosphate ratio of about 3-6 (N:P), or cells are treated with segRNA in LNPs formulated with an amine-to-RNA-phosphate ratio of about 3-6 (N:P) and AAV vector encoding Cas9 protein.

### Example 13

### Anti-HBV in Chimeric Mice

The antiviral assay in HBV infected chimeric mice is performed according to a reported procedure except Cas9-segRNA RNP complexes or their conjugates are administrated instead of small interfering RNAs (Thi et al. ACS Infec. Dis. 2019, 5, 725-737, the entire disclosure of which is incorporated herein by reference.). All animals are bred under specific pathogen-free conditions in accordance with the ethical guidelines set forth by the National Institutes of Health for care of laboratory animals. The cDNA-uPA/SCID (cDNA-uPA (+/wt)/SCID (+/+)) hemizygote mice are generated as described. Cryopreserved human hepatocytes (2-year-old female, Hispanic, BD195, BD Biosciences) are transplanted into 2 week-old hemizygous cDNA-uPA/SCID mice via the spleen under anesthesia. The human hepatocytes are allowed to expand for 10-12 weeks and the replacement index are tested by measuring human albumin (h-Alb) in blood collected from tail vein using clinical chemistry analyzer (BioMajesty Series JCA-BM6050, JEOL Ltd.) with latex agglutination immunonephelometry (LZ Test “Eiken” U-ALB, Eiken Chemical Co., Ltd.). Male chimeric mice with more than 7.0 mg/mL h-Alb concentration in blood are judged as PXB mice whose replacement index is more than 70%.

PXB mice (>70% replacement index, 13-15 weeks old) are infected with HBV by intravenous injection through the tail vein with 1×10⁵ copies of HBV containing serum from previously infected animals. Eight weeks post infection, animals with HBV DNA titers greater than 1.0×10⁶ copies/mL and h-Alb greater than 7.0 mg/mL are selected (n=5 per group). Cas9-segRNA complexes are dosed via the lateral tail vein in a volume of 0.2 mL per animal. Animals are euthanized at various time points by exsanguination under isoflurane anesthesia. Liver tissue is collected from the median or left lateral lobe from each animal for DNA extraction and for NGS. Editing efficiency and off-targets are determined as described (Finn et al. Cell Reports 2018, 22, 2227-2235; Tsai et al. Nat. Methods 2017, 14, 607-614).

Blood is collected into serum separator tubes. Serum HBV DNA is assayed by qPCR and serum HBsAg measured by chemiluminescence enzyme immunoassay (ARCHITECT, Abbott). Serum HBeAg is also assessed using a chemiluminescence enzyme immunoassay (ARCHITECT, Abbott). Liver total and 3.5 kb HBV (pg)RNA at day 42 (study termination) are analyzed by Quantigene 2.0 b DNA assay (Affymetrix), and data is normalized to human glyceraldehyde-3-phosphate dehydrogenase (GAPDH) mRNA. Immunohistochemical analysis for HBcAg is conducted on liver sections at day 42.

Alternatively, segRNA and mRNA encoding Cas9 protein (segRNA/mRNA˜10:1) are administrated either as a mixture or sequentially in LNPs formulated with an amine-to-RNA-phosphate ratio of about 3-6 (N:P), or AAV vector encoding Cas9 protein and segRNA in LNPs formulated with an amine-to-RNA-phosphate ratio of about 3-6 (N:P) are administrated sequentially.

In some experiments, a mixture of segRNAs or their conjugates targeting different loci, and/or variants of HBV genes are used (Multiplexing editing. See Example 11.).

### Example 14

### RNA Editing with Segmented gRNAs in ADAR-Expressing 293 Cells

RNA editing in ADAR-expressing 293 cells is performed according to a reported procedure (See, e.g., Merkle et al. Nature Biotech. 2019, 37, 133-138, the entire disclosure of which is incorporated herein by reference.). Segmented gRNA ASO (5 pmol/well unless stated otherwise) and Lipofectamine 2000 (0.75 μL/well) are each diluted with OptiMEM to a volume of 10 μL in separate tubes. After 5 min, the two solutions are mixed and 100 μL cell suspension (5×10⁴ cells) in DMEM plus 10% FBS plus 10 ng/mL doxycycline is added to the transfection mixture inside 96-well plates. Twenty-four hours later, cells are harvested for RNA isolation and sequencing.

What is claimed is:

1. Method for preparation of segmented nucleic acids joined by triazole linkers, comprising:
a) Synthesis of segment 1 of 8-200 nt in length containing azido modification at its 3′-end;
b) Synthesis of segment 2 of 8-200 nt in length containing an alkyne at its 5′-end or at a position close to its 5′-end, and an amino at its 3′-end or a position close to its 3′-end;
c) Conjugation of said segment 1 and 2 by reaction between said azido and alkyne to form a two-segmented nucleic acid linked by the resulting triazole;
d) Transformation of said amine of said two-segmented nucleic acid in step c) into an azido;
e) Conjugation of azido two-segmented nucleic acid in d) to another segment between said azido and an alkyne in said another segment.
f) Optionally, step d) and e) can be repeated as needed wherein said another segment in step e) is modified to contain an amine;
g) Separate the segmented nucleic acid from unreacted shorter segment and chemical reagents.

2. Said Synthesis of segment 1 in claim 1, step a) is performed on an alcohol attached to a solid support, wherein said alcohol is substituted with an azido group, subsequent global deprotection gives segment 1 containing azido modification at its 3′-end.

3. Said transformation in step d) of claim 1 is a diazotransfer reaction with fluorosulfuryl azide.

4. (canceled)

5. Said transformation in step d) of claim 1 is an amide formation with a ligation function-substituted NHS ester, and e) of claim 1 is replaced with:
e) Conjugation of resulting two-segmented nucleic acid in d) to another segment between said ligation function and a compatible ligation function in said another segment.

6. Method for preparation of 5′-alkynyl, 3′-aimino nucleic acid of claim 1, step b), comprising:
a) Extension with nucleotide phosphoramidites at an alcohol attached to a solid support, wherein said alcohol is substituted with a protected amino group;
b) Addition of 5′-alkynyl modifier to the detritylated oligonucleotide on solid support;
c) Cleavage of solid support and global deprotection give a 5′-alkynyl, 3′-amino nucleic acid, wherein the amino protecting group is removed after cleavage of cyanoethyl phosphate esters.

7. Said segmented nucleic acid of claim 1 is a guide RNA as a component of a CRISPR-Cas RNP complex.

8. Said guide RNA of claim 7, its 3′-terminal segment comprises a DNA segment at its 3′-terminus of 18-200 nt in length.

9. Said 3′-terminal segment of claim 8, the 3′-terminal segment further comprises an RNA segment of 3′-end of a tracrRNA covalently tethered to the 5′-end or 3′-end of said DNA segment.

10. Said guide RNA of claim 7, its 5′-terminal segment comprises an ssDNA segment at its 5′-terminus of 18-200 nt in length.

11. Said segmented nucleic acid of claim 1 is a ribozyme.

12. Said segmented nucleic acid of claim 1 is an aptamer.

13. Said segmented nucleic acid of claim 1 is a guide RNA of human ADAR1 or ADAR2.

14. Said segmented nucleic acid of claim 1 is an RNA conjugate.

15. Method of claim 1 for preparation of three-segmented nucleic acids joined by triazole linkers, comprising:
a) Synthesis of segment 1 of 8-200 nt in length containing alkynyl modification at its 3′-end;
b) Synthesis of segment 2 of 8-200 nt in length containing an amino at its 5′-end or at a position close to its 5′-end, and an azido at its 3′-end or a position close to its 3′-end;
c) Synthesis of segment 3 of 8-200 nt in length containing alkynyl modification at its 5′-end;
d) Conjugation of said segment 2 and 3 by reaction between said azido and alkyne to form a two-segmented nucleic acid linked by the resulting triazole;
e) Transformation of said amine of said two-segmented nucleic acid in step d) into an azido by a diazotransfer reaction with fluorosulfuryl azide;
f) Conjugation of azido two-segmented nucleic acid in e) to segment 1 between said azido and the alkyne in segment 1;
g) Separate the segmented nucleic acid from unreacted shorter segment and chemical reagents.

16. Method of claim 1 for preparation of three-segmented nucleic acids joined by triazole linkers, comprising:
a) Synthesis of segment 1 of 8-200 nt in length containing alkynyl modification at its 3′-end;
b) Synthesis of segment 2 of 8-200 nt in length containing an azido at its 5′-end or at a position close to its 5′-end, and an amino at its 3′-end or a position close to its 3′-end;
c) Synthesis of segment 3 of 8-200 nt in length containing alkynyl modification at its 5′-end;
d) Conjugation of said segment 1 and 2 by reaction between said azido and alkyne to form a two-segmented nucleic acid linked by the resulting triazole;
e) Transformation of said amine of said two-segmented nucleic acid in step d) into an azido by a diazotransfer reaction with fluorosulfuryl azide;
f) Conjugation of azido two-segmented nucleic acid in e) to segment 3 between said azido and the alkyne in segment 3;
g) Separate the segmented nucleic acid from unreacted shorter segment and chemical reagents.

17. Method of claim 1 for preparation of three-segmented nucleic acids joined by a triazole linker and an amide linker, comprising:
a) Synthesis of segment 1 of 8-200 nt in length containing alkynyl modification at its 3′-end;
b) Synthesis of segment 2 of 8-200 nt in length containing an azido at its 5′-end or at a position close to its 5′-end, and an amino at its 3′-end or a position close to its 3′-end;
c) Synthesis of segment 3 of 8-200 nt in length containing a phosphine at its 5′-end;
d) Conjugation of said segment 1 and 2 by reaction between said azido and alkyne to form a two-segmented nucleic acid linked by the resulting triazole;
e) Transformation of said amine of said two-segmented nucleic acid in step d) into an azido by a diazotransfer reaction with fluorosulfuryl azide;
f) Conjugation of azido two-segmented nucleic acid in e) to segment 3 between said azido and the phosphine in segment 3;
g) Separate the segmented nucleic acid from unreacted shorter segment and chemical reagents.

18. Method for preparation of segmented nucleic acid conjugates by sequential ligations, comprising:
a) Transformation of an amine into an azido by a diazotransfer reaction with fluorosulfuryl azide after the previous ligation step, wherein a conjugate is formed, and before next ligation via newly formed azido;
b) ligation of azido segmented conjugate in a) to a next chemical moiety between its alkyne or phosphine and the newly formed azido in said conjugate.

19. Said diazotransfer reaction with fluorosulfuryl azide in claim 18 is replaced by amide bond formation with an azido NHS ester.

20. Method for preparation of segmented nucleic acid conjugates by sequential ligations, comprising:
a) Preparation of a 5′ or 3′ amino segmented nucleic acid by sequential ligations;
b) conjugation of said 5′ or 3′ amino segmented nucleic acid in a) to a carboxylic acid or NHS ester by formation of an amide.

21. Method of claim 1 for preparation of three-segmented nucleic acids joined by triazole linkers, comprising:
a) Synthesis of segment 1 of 8-200 nt in length containing azido modification at its 3′-end;
b) Synthesis of segment 2 of 8-200 nt in length containing an alkynyl at its 5′-end or at a position close to its 5′-end, and an amino at its 3′-end or a position close to its 3′-end;
c) Synthesis of segment 3 of 8-200 nt in length containing alkynyl modification at its 5′-end;
d) Conjugation of said segment 1 and 2 by reaction between said azide and alkyne to form a two-segmented nucleic acid linked by the resulting triazole;
e) Transformation of said amine of said two-segmented nucleic acid in step d) into an azido by a diazotransfer reaction with fluorosulfuryl azide;
f) Conjugation of azido two-segmented nucleic acid in e) to segment 3 between said azido and the alkyne in segment 3;
g) Separate the segmented nucleic acid from unreacted shorter segment and chemical reagents.
